(19) United States
(12) Patent Application Publication
(10) Pub. No.: US 2026/0000471 A1
(43) Pub. Date: Jan. 1, 2026

(54) CATHETER ASSEMBLY MOTION DETECTION INCLUDING PROLAPSE DETECTION

(71) Applicant: Imperative Care, Inc., Campbell, CA (US)

(72) Inventors: Konrad Marek Guenter Leibrandt, Corona, CA (US); Steve Burion, San Francisco, CA (US); Matthew Gunther, Campbell, CA (US); Richard Leparmentier, Campbell, CA (US); Mingyi Zheng, San Jose, CA (US)

(21) Appl. No.: 19/247,721

(22) Filed: Jun. 24, 2025

Related U.S. Application Data
(60) Provisional application No. 63/664,344, filed on Jun. 26, 2024.

(51) Int. Cl. A61B 34/30; A61B 34/00; A61B 34/10; A61B 90/00; G06T 7/70; G16H 40/63

(52) U.S. Cl. CPC A61B 34/30; A61B 34/10; A61B 34/25; A61B 34/76; A61B 90/37; A61B 90/39; G06T 7/70; G16H 40/63; A61B 2034/252; A61B 2034/301; A61B 2090/376; A61B 2090/3966; G06T 2207/10121; G06T 2207/30021

(57) ABSTRACT

A system for robotically controlling interventional devices can detect catheter prolapse. The system can estimate a catheter position based at least in part on the imaging of a patient. The system can compare changes in the image-based catheter position to data provided by a catheter drive system. Based on this comparison, the system can determine whether catheter prolapse has occurred or the degree to which catheter prolapse has occurred. Methods of detecting and predicting catheter prolapse are also disclosed.

### BACKGROUND

### Field

The present application relates to neurovascular procedures, and more particularly, to catheter assemblies and robotic control systems for neurovascular site access.

### Description of the Related Art

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neurointerventions. Neuro interventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due to energy storage and release caused by frictional interplay between coaxial shafts and the patient's vasculature. Supra-aortic access necessary to reach the neurovasculature is challenging to achieve, especially Type III arches. Once supra-aortic access is achieved, adapting the system for neurovascular treatments is time consuming and requires guidewire and access catheter removal and addition of a procedure catheter (and possibly one or more additional catheters) to the guidewire/catheter stack.

Thus, there remains a need for a supra-aortic access and neurovascular site access system that addresses some or all these challenges and increases the availability of neurovascular procedures. Preferably, the system is additionally capable of driving devices further distally through the supra-aortic access to accomplish procedures in the intracranial vessels.

### SUMMARY

There is provided a system for detecting catheter or guidewire prolapse, the system including: one or more processors that can receive a plurality of images from an imaging system and drive system sensor data from a catheter drive system. The one or more processors can further: determine a first catheter position based at least in part on a first of the plurality of images, the first catheter position including at least a first endpoint and a first centerline; determine a second catheter position of based at least in part on a second of the plurality of images, the second catheter position including at least a second endpoint and a second centerline; determine a visual position change based on a comparison of the first endpoint to the second endpoint; determine an expected motion based at least in part of the drive system sensor data and a drive calibration, where the expected motion corresponds to drive system user input over a duration spanning the first of the plurality of images to the second of the plurality of images, the drive calibration including an expected movement distance corresponding to a calibration input; determine a difference between the visual position change and the expected motion; and cause an alert to a user of the system if the difference exceeds a prolapse threshold, the alert indicative of occurrence of catheter or guidewire prolapse.

In some examples, the prolapse threshold is a relative threshold of 5% difference between movement indicated from the images and from the drive system data. In some examples, the alert includes a user graphic. In some further examples, the user graphic includes a prompt, where the prompt requires user input to allow further input to the catheter drive system. In some examples, the alert includes haptic feedback to a controller of the drive system. In some examples, the system further includes the catheter drive system. In some examples, the system further includes the imaging system. In some examples, the first endpoint and second endpoint correspond to an endpoint of a guidewire in the first and second of the plurality of images. In some examples, the first endpoint and second endpoint correspond to an endpoint of a catheter in the first and second of the plurality of images. In some examples, the one or more processors can execute programming to for computer vision analysis of at least some of the plurality of images. In some examples, the one or more processors can further: compare the first centerline to the second centerline to determine a curvature change; and cause a buckling alert to a user of the system if the curvature change exceeds a curvature threshold. In some examples, at least some of the plurality of images include an image of a radio-opaque portion of the catheter or guidewire, and the processor can validate at least one of the determined first catheter position and second catheter position against a position of the radio-opaque portion of the catheter or guidewire. In some examples, the first endpoint is positioned along the first centerline, and where the second endpoint is positioned along the second centerline.

There is provided a method of robotically controlling a catheter, the method including: receiving a plurality of images from an imaging system and drive system sensor data from a catheter drive system; determining a first catheter position based at least in part on a first of the plurality of images, the first catheter position including at least a first endpoint and a first centerline; determining a second catheter position of based at least in part on a second of the plurality of images, the second catheter position including at least a second endpoint and a second centerline; determining a visual position change based on a comparison of the first endpoint to the second endpoint; determining an expected motion based at least in part of the drive system sensor data and a drive calibration, where the expected motion corresponds to drive system user input over a duration spanning the first of the plurality of images to the second of the plurality of images, the drive calibration including an expected movement distance corresponding to a calibration input; determining a difference between the visual position change and the expected motion; and causing an alert if the difference between the visual position change and the expected motion exceeds a prolapse threshold, the alert indicative of occurrence of catheter or guidewire prolapse.

In some examples, the prolapse threshold is 5%. In some examples, the method further includes blocking user input to a catheter drive system if the difference between the visual position change and the expected motion exceeds the prolapse threshold. In further examples, the alert includes a prompt, and where the prompt requires user input to allow further input to the catheter drive system. In some examples, the first endpoint and second endpoint correspond to an endpoint of a guidewire or a catheter in the first and second of the plurality of images. In some examples, the method further includes: comparing the first centerline to the second centerline to determine a curvature change; and causing a buckling alert if the curvature change exceeds a curvature threshold. In some examples, the alert includes haptic feedback to a controller of the drive system. In some examples, at least some of the plurality of images include an image of a radio-opaque portion of the catheter or guidewire, and the method further includes validating at least one of the determined first catheter position and second catheter position against a position of the radio-opaque portion of the catheter or guidewire. In some examples, the first endpoint is positioned along the first centerline, and the second endpoint is positioned along the second centerline.

There is provided in accordance with one aspect of the present disclosure a system for detecting catheter prolapse. The system includes a processor that can receive image data from an imaging system and drive system sensor data from a catheter drive system. The processor can further: estimate a catheter position based at least in part on the image data; determine a catheter position change based on a comparison of the catheter position to a previous catheter position; and determine, based on a comparison of the catheter position change to the drive system sensor data, whether a catheter prolapse has occurred.

In some examples, the processor may be able to determine, based on a comparison of the catheter position change to the drive system sensor data, the degree to which catheter prolapse has occurred. The system may additionally include the catheter drive system, and the catheter drive system can actuate movement of the catheter; and the imaging system. The imaging system may be able to image the body of a patient and to transmit image data to the processor.

The processor may be able to output to a display an indication of catheter prolapse in response to a determination that catheter prolapse has occurred. The system may further include the display. The display can indicate catheter prolapse to a user.

The processor may be able to detect background movement in the image data. The processor may be able to process the image data. The processor may further be able to process the image data by running the image data through a vision encoder. The imaging data may include angiograms. The processor may include a neural network. The neural network may further be able to determine the catheter position. The processor may be able to detect a centerline of the catheter and a tip of the catheter. The processor may be able to determine catheter prolapse based on a threshold difference between the catheter position change and the drive system sensor data.

The catheter position change may be based at least in part on a change in a position of the catheter tip. The catheter position change may be based at least in part on a change in a position of the catheter centerline. The processor may be local to the imaging system and the catheter drive system. The system may further include a control system and a display, the control system and display in electrical communication with the processor, the control system and display being remote to the processor.

The catheter drive system may be capable of actuating movement of the catheter within a blood vessel of a patient. The catheter drive system may be capable of actuating movement of a plurality of catheters, the plurality of catheters including one or more procedure catheters. The plurality of catheters may include an access catheter.

There is also provided a method of robotically controlling a catheter. The method includes: receiving a plurality of images, where at least some of the plurality of images capture the catheter; causing movement of the catheter while at least some of the plurality of images are generated; determining a first catheter position and a second catheter position based at least in part on the plurality of images; determining a catheter position change based on a comparison of the first position and the second position; and determining, based on a comparison of the catheter position change to the caused movement, whether a catheter prolapse has occurred.

The method may include determining, based on a comparison of the catheter position change to the caused movement, the degree to which catheter prolapse has occurred. The method may further include notifying a user the degree to which the catheter prolapse has occurred. The method may include notifying a user that the catheter prolapse has occurred. The method may include detecting background movement in the plurality of images and excluding at least one of the plurality of images based on a detection of background movement. The method may include imaging at least a portion of a body of a patient. The method may include processing the plurality of images to generate digital subtraction angiograms.

The first catheter position may be a first catheter tip position and the second catheter position may be a second catheter tip position. The first catheter position may be a first catheter centerline position and the second catheter position may be a second catheter centerline position. The method may include determining whether prolapse has occurred based at least in part on a threshold difference between the catheter position change and the driving. The first catheter position may be based on a first image of the plurality of images and second catheter position is based on a second image of the plurality of images. The first image and the second image may be consecutive images of the plurality of images.

There is also provided a method of detecting catheter prolapse. The method may include: receiving image data and catheter drive system sensor data; determining a catheter position change based at least in part on the image data; and determining, based on a comparison of the catheter position change to the catheter drive system sensor data, whether a catheter prolapse has occurred. The method may further include determining, based on a comparison of the catheter position change to drive system sensor data, the degree to which catheter prolapse has occurred.

There is also provided a system for predicting prolapse of a catheter. The system includes a processor configured to receive image data from an imaging system and drive system sensor data from a catheter drive system, the processor capable of detecting, based on the image data, a catheter centerline, determining a catheter tension based on a curvature of the catheter centerline, and determining a likelihood of catheter prolapse based on the catheter tension.

The system may further include: the catheter drive system, the catheter drive system able to actuate movement of the catheter; and the imaging system, the imaging system able to image the body of a patient and transmit image data to the processor. The processor may be able to output to a display an indication of the likelihood of catheter prolapse. The system may further include the display, the display able to indicate the likelihood of catheter prolapse to a user.

The processor may be able to output an alert if the likelihood of catheter prolapse is above a threshold. The image data may include angiograms. The processor may be local to the imaging system and the catheter drive system. The processor may be able to detect background movement in the image data. The processor may be able to process the image data. The processor may include a neural network configured to detect the catheter centerline. The processor may be local to the imaging system and the catheter drive system. The catheter drive system may be able to actuate movement of the catheter within a blood vessel of a patient.

There is also provided a method of predicting prolapse of a catheter. The method includes: receiving a plurality of images from an imaging system and movement data from a catheter drive system, where at least some of the plurality of images capture the catheter; determining, based on the plurality of images, a catheter centerline; determining a catheter tension based on a curvature of the catheter centerline; and determining a likelihood of catheter prolapse based on the catheter tension.

The method may include outputting an alert if the likelihood of catheter prolapse is above a threshold. The method may include generating angiograms based on the plurality of images, where the determining a catheter centerline is based on the angiograms. The method may include detecting background movement in the plurality of images and excluding at least one of the plurality of images based on a detection of background movement. The method may include imaging at least a portion of a body of a patient.

### BRIEF DESCRIPTION OF THE DRAWINGS

FIG. 1 is a schematic perspective view of an interventional setup having an imaging system, a patient support table, and a catheter drive system in accordance with the present disclosure.

FIG. 2A is a block diagram of an interventional setup capable of detecting and/or predicting catheter prolapse.

FIG. 2B illustrates a system diagram of an embodiment of a control system.

FIG. 3A illustrates a side elevational schematic view of an interventional device assembly for supra-aortic access and neuro-interventional procedures.

FIGS. 3B-3F depict an example sequence of steps of introducing a catheter assembly configured to achieve supra-aortic access and neurovascular site access.

FIGS. 4A and 4B schematically illustrate a sensor for measuring elastic forces at the magnetic coupling between the hub and corresponding carriage of the catheter drive system.

FIG. 4C schematically illustrates a dual encoder torque sensor for use by a catheter drive system of the present disclosure.

FIG. 5 schematically diagrams a method of processing image data and catheter drive data to detect and/or predict catheter prolapse.

FIGS. 6 and 7 schematically diagram methods to perceive a guidewire/catheter stack in images taken by the imaging system.

FIG. 8A is an image of a guidewire/catheter stack in a model blood vessel.

FIG. 8B is an image showing output of centerline detection based on the image of FIG. 8A.

FIG. 8C is an image showing output of endpoint/transition point detection based on the image of FIG. 8A.

FIG. 8D is an image showing segmentation of the centerline of FIG. 8B based on the endpoint/transition point detection of FIG. 8C.

FIG. 8E is an image showing an overlay of the guidewire/catheter stack perceived in FIGS. 8B-8D on mapped vasculature.

FIGS. 9 and 10 schematically diagram methods for detecting catheter prolapse based on image data and drive system data.

### DETAILED DESCRIPTION

In certain aspects, a system is provided for detecting and/or predicting catheter prolapse for a robotic catheter drive system. The system can detect and/or predict catheter prolapse based on images of a patient and data from the robotic catheter drive system. The robotic catheter drive system may allow for precise control movements of the catheter and/or guidewire through a patient's vasculature. Catheter prolapse can cause physicians to lose access to certain blood vessels, for example if a guidewire tip exits a blood vessel due to catheter prolapse. As such, early detection and/or prediction of catheter prolapse may be advantageous to allow healthcare providers to better navigate a patient's vasculature

Additionally, the robotic system does not transmit the exact same tactile feedback as a healthcare provider would have while manually using a catheter and/or guidewire on a patient. In practiced hands, such tactile feedback may help indicate occurrence of catheter prolapse. For use of a robotic catheter drive system, however, it may be advantageous to detect and/or predict catheter prolapse using information available from patient imaging and the robotic catheter drive system. For such systems, it would be difficult or impossible for a user to notice occurrence of prolapse, due in part to the lack of tactile feedback relative to manual use of a catheter and/or guidewire. Accordingly, such automatic prolapse detection represents an improvement to the safety and usability of remote catheter drive systems.

In certain aspects, methods are provided for detecting and/or predicting catheter prolapse.

### Overview of System

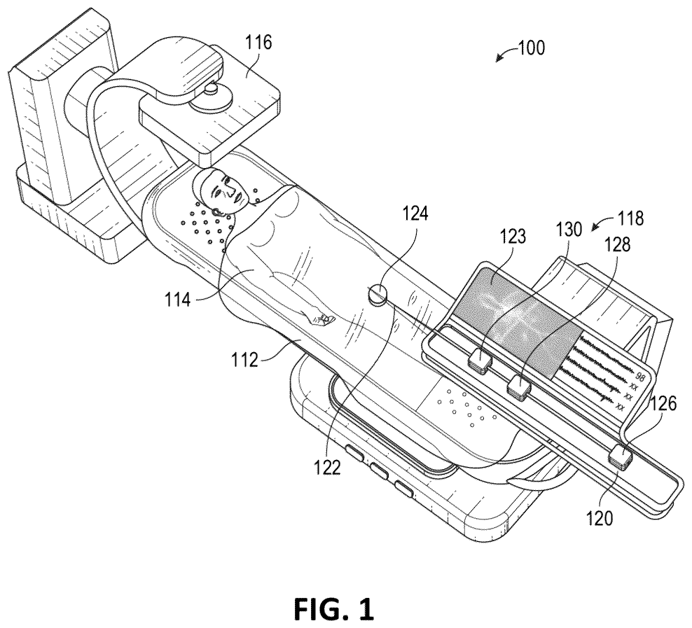

FIG. 1 is a schematic perspective view of an interventional setup 100 having a patient support table 112 for supporting a patient 114. An imaging system 116 may be provided, along with a catheter drive system 118 (also referred to herein as a drive system 118) in accordance with the present disclosure. Interventional setups and methods are discussed in U.S. Pub No. 2024/0042124, entitled FLUIDICS CONTROL SYSTEM FOR MULTI CATHETER STACK, filed Aug. 2, 2022; U.S. Pub. No. 2024/0032949, entitled METHOD OF SUPRA-AORTIC ACCESS FOR A NEUROVASCULAR PROCEDURE, filed Aug. 1, 2022; U.S. Pub. No. 2024/0180659, entitled METHOD FOR ROBOTICALLY CONTROLLING SUBSETS OF INTERVENTIONAL DEVICE ASSEMBLY, filed Nov. 30, 2023; U.S. Pub. No. 2024/0382668, entitled FLUIDICS CONTROL SYSTEM FOR MULTI CATHETER STACK, filed May 16, 2024; U.S. Pub. No. 2025/0032700, entitled SYSTEM FOR REMOTE MEDICAL PROCEDURE, filed Jul. 25, 2024; U.S. application Ser. No. 18/986,519, entitled ROBOTIC HUB ASSEMBLY, filed Dec. 18, 2024; U.S. App. No. 63/664,547, entitled INSERT CATHETER FOR ROBOTIC MULTI-CATHETER ASSEMBLY, filed Jun. 26, 2024; U.S. application Ser. No. 19/228,455, entitled DRIVE TABLE, filed Jun. 4, 2025; and U.S. application Ser. No. 19/228,468, METHOD FOR ROBOTICALLY CONTROLLING INTERVENTIONAL DEVICE ASSEMBLY, filed Jun. 4, 2025; each of which are incorporated by reference in their entireties.

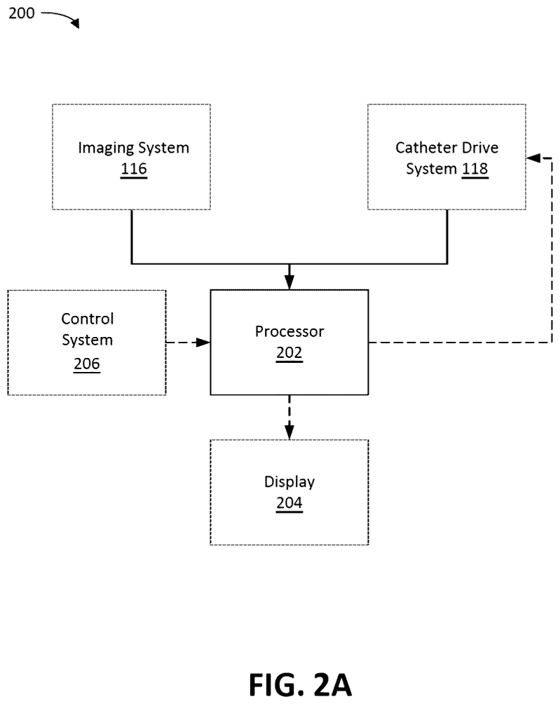

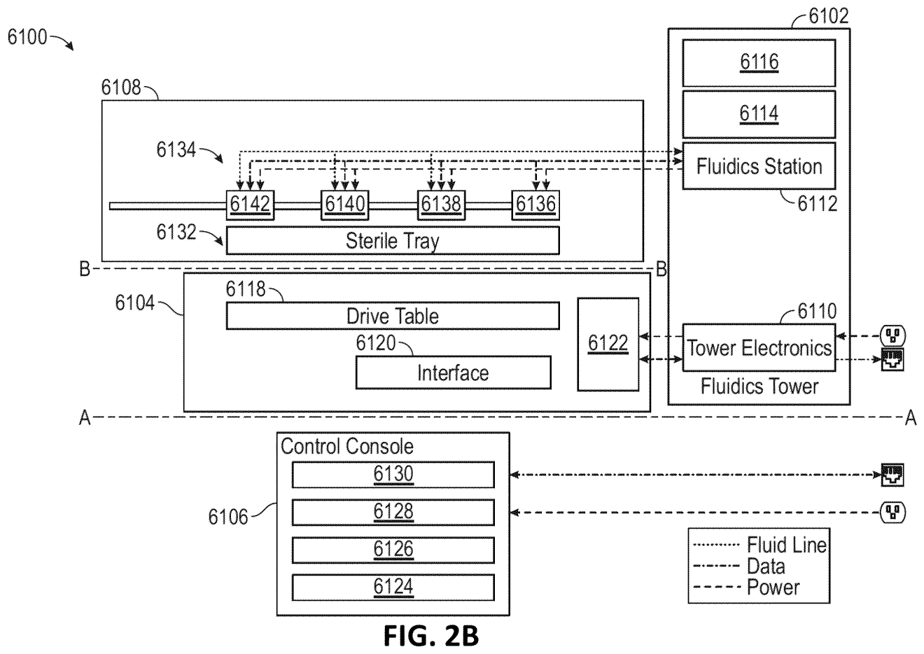

FIG. 2A is a block diagram of a system in accordance with the present disclosure. The processor 202 can receive signals and/or data from the imaging system 116 and the catheter drive system 118. Optionally, the processor 202 can output to a display 204. The processor 202 may be local to (e.g., within the same room as the patient or at the bedside of the patient) or remote to (e.g., in a different room as or in a different building as) the imaging system 116 and the catheter drive system 118. Optionally, the processor 202 can receive input from a control system 206 and transmit the input to the catheter drive system 118. In such aspects, movement of the catheter drive system 118 may be controlled by the processor 202 and the control system 206. Example controllers and control systems are discussed in U.S. patent application Ser. No. 18/525,267. Each of the control system 206 and the display 204 may be remote to the patient 114, the imaging system 116, and the catheter drive system 118. The control system 206 and the display 204 may each be a part of a surgical console that can allow a physician to operate the catheter drive system 118 and view live images of the patient.

The processor 202 may be capable of communicating with the imaging system 116, the catheter drive system 118, the display 204, and the control system 206 over any suitable network. The network may be a wired network, a wireless network, or combination thereof. Additionally, the network may be a personal area network, a local area network, a wide area network, a cable network, a satellite network, a cellular telephone network, or a combination thereof. Protocols and components for internet-based communication or any of the other aforementioned types of networks are known to those skilled in the art of computer communications. The network has sufficient bandwidth to stream images from the imaging system 116 to the processor 202 and transmit commands from the processor 202 to the catheter drive system 118. The network has sufficient bandwidth to transmit results of the prolapse detection and prediction algorithms from the processor 202 to the display 204 and/or control system 206.

FIG. 2B illustrates a system diagram of an example of a medical device operation system 6100. The system diagram shows fluid and electrical connectivity between the subsystems of the medical device operation system 6100. The medical device operation system 6100 may include a fluidics tower 6102, a robotic drive system 6104, a control system 6106, and one or more interventional devices 6108.

The fluidics tower 6102 may be a housing or console including a fluidics management system for controlling the administration or removal of contrast, saline and/or bodily fluids to and/or from an interventional device. The fluidics tower 6102 may further include an electronics tower 6110, a fluidics station (or “system”) 6112, a monitor 6114, and one or more communication devices 6116. Although illustrated in FIG. 2B as part of the fluidics tower 6102, in other embodiments the electronics tower 6110 may be housed separately from the fluidics tower 6102 while still being in communication with the fluidics tower 6102, the interventional devices 6108, the robotic drive system 6104, and the control console system.

The electronics tower 6110 may be a housing configured to contain system electronics such as one or more processors and memory. The one or more processors and memory may be organized into one or more computer devices. The electronics tower 6110 may include a power cord configured to be operatively coupled to a power source, such as a battery, a generator, or an outlet. In some embodiments, the electronics tower 6110 may draw power from a source providing 110/220 volts of alternating current (VAC) power.

The system electronics may be a central hub for the medical device operation system 6100 interconnecting the electronic devices from other components as described in greater detail below. The system electronics of the electronics tower 6110 may be configured to transmit and receive electronic signals and/or data to operate components of the medical device operation system 6100. The electronics tower 6110 may include means for connecting to other devices. The electronics tower 6110 may transmit and/or receive electronic signals and/or data wirelessly or over a wired connection. In some embodiments, the electronics tower 6110 may include an ethernet port for connecting the system electronics to a network. In some embodiments, the electronics tower 6110 may include one or more ports for tethering to nearby electronic devices via a wired connection. For example, the electronics tower 6110 may have ports to run cables between the fluidics tower 6102 and the robotic drive system 6104 and/or control system 6106. Alternatively, the electronics tower 6110 may be configured to connect wirelessly to nearby electronic devices. For example, the electronics tower 6110 may include a personal area network (PAN) module, such as Bluetooth®, or other network capabilities to transmit data wirelessly.

In some embodiments, electronic signals and/or data may include instructions for a system, subsystem, component, or device to perform a particular task. Additionally and/or alternatively, electronic signals and/or data may include indicators or data measured from sensors for processing by a computing device.

In some embodiments, the electronics tower 6110 may connect to other devices over a communication network (“network”). The network may cover a small geographic area such as a particular room or building, a medium geographic area such as a city, or a large geographic area so long as there is access to a network. For example, the network may be a local area network (LAN) or wireless local area network (WLAN) including a series of devices linked together to form a network within a hospital or clinic. Alternatively, the network may be a wide-area network (WAN) including a series of devices linked together to form a network within a medical campus including two or more buildings. Alternatively, the network may be an intranet or internet for providing global connectivity. Connecting over the network advantageously connects the operating room to physicians located around the world, including experts located across the nation or in other countries, without requiring the physician to travel to the operating room. This advantageously connects patients to physicians without the time or cost required for the physician to physically travel to the operating room. In some procedures, every minute of delay before a procedure is performed can increase the chance of a bad outcome, and thus such surgical systems can help mitigate damage to the patient due to a delay in starting the surgical procedure.

The fluidics system 6112 may include one or more subsystems including one or more containers, one or more tubes, and one or more pumps. The subsystems may be divided and organized into a contrast subsystem, a saline subsystem, and/or an aspiration (or “vacuum”) subsystem.

A contrast subsystem may be configured for supplying contrast to a patient. The contrast subsystem may include one or more containers for storing and supplying contrast, one or more fluid communication channels (“tubes”), one or more valves, and a high-pressure pump.

A saline subsystem may be configured for supplying saline to a patient. The saline subsystem may similarly include one or more containers for storing and supplying saline, one or more tubes, one or more valves, and one or more pumps.

An aspiration subsystem may be configured for removing biological material from a patient. The aspiration subsystem may include one or more containers, one or more tubes, one or more valves, and a vacuum pump.

The one or more pumps and containers of the contrast, saline, and aspiration subsystems may be contained with the fluidics tower 6102. The one or more tubes of the contrast, saline, and aspiration subsystems may extend out of the fluidics tower 6102 for interacting with and coupling to other devices.

The monitor 6114 may be any electronic visual computer display (or displays) that includes a screen and circuitry configured to interpret electronic signals to display one or more images. For example, the monitor 6114 may include an imaging window, a speed indicator, a rotational indicator, an axial position bar, a telescopic position window, one or more axial position indicators, and/or other graphical user interfaces or windows. In some embodiments, the monitor 6114 may be configured to display fluoroscopic images, catheter data, fluidics information (e.g., information relating to a contrast injection subsystem including its current operation status, information relating to a saline subsystem including its current operation status, and/or information relating to a aspiration subsystem including its current operation status) including current state information) providing saline, providing vacuum for aspiration), and patient data including vital signs. In some embodiments, the monitor 6114 may be the display 204 described above.

The one or more communication devices 6116 may be one or more microphones, one or more cameras, and/or one or more audio output devices such as a speaker and/or a headset.

The fluidics system 6112, the monitor 6114, and the one or more communication devices 6116 may be electrical communication with the electronics tower 6110 and configured to receive and/or transmit electronic signals and/or data therebetween. In some embodiments, the electronic signals and/or data may include instructions to activate one or more pumps and/or one or more valves of the fluidics system 6112. For example, the instructions may direct the fluidics system 6112 to provide saline and/or contrast to the one or more interventional devices 6108. In some embodiments, the data may include video and/or audio inputs and audio outputs for the monitor 6114 and one or more communication devices 6116. For example, the data may be one or more images to be displayed on the monitor 6114 and/or audio-visual data captured by the one or more communication devices 6116.

The fluidics tower 6102 may be further configured to be operatively coupled with the one or more interventional devices 6108. In some embodiments, the fluidics system 6112 may be mechanically coupled to and/or in fluid communication with the one or more interventional hubs 6134. Accordingly, activating the fluidics system 6112 may provide contrast, saline, and/or suction to the interventional hubs 6134 and corresponding interventional devices.

The robotic drive system 6104 may include a plurality of components to drive one or more access systems such as catheters and guidewires during a procedure. The robotic drive system 6104 may be the catheter drive system 118 described above. The robotic drive system 6104 may include a drive table 6118, an interface 6120, and a joint setup 6122.

The drive table 6118 may support the one or more disposable devices 6108 (e.g., a catheter) configured to be advanced to access a patient for performing a surgical procedure and/or for introducing saline, contrast media or therapeutic agents, or providing aspiration. The drive table 6118 may further support a sterile barrier.

The drive table 6118 may be the support table 112 described above. The drive table 6118 may be positioned over or alongside a patient, and configured to axially advance, retract, and in some cases rotate two or three or more different concentrically oriented intravascular devices of an interventional device assembly. The drive table 6118 may include electronics and motors for controlling the location of the interventional devices and actuation of fluidics components.

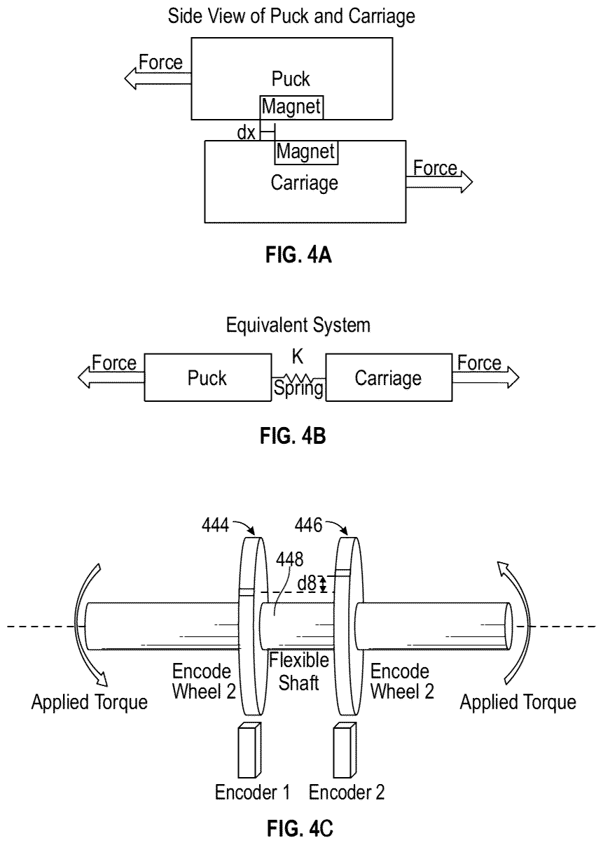

In some embodiments, the drive table 6118 may include one or more hub adapters. The one or more hub adapters may include the drive magnets described herein, for example with reference to FIGS. 4A and 4B. Movement of the drive magnets may be driven by a drive system carried by the drive table 6118. Movement of the drive magnets may be configured to drive one or more interventional hubs 6134 of the one or more disposable devices 6108. The drive table 6118 and the one or more disposable devices 6108 may be separated such that the one or more interventional hubs 6134 may not mechanically couple to the drive table 6118 as shown by axis B-B.

The interface 6120 may be any device configured to interact with and/or display information to personnel locally situated within an operating room during a procedure, such as a bedside user. For example, a bedside user may be nurse or surgical technician staffed within the operating room. The interface 6120 may include an imaging window, a speed indicator, a rotational indicator, an axial position bar, a telescopic position window, and/or one or more axial position indicators. The interface 6120 may be the display 204 described above configured to display fluoroscopic images, catheter data, pressure values of the fluidics system, and/or other patient data. In some embodiments, the interface 6120 may be a touchscreen device such as a tablet computer. The interface 6120 may display information to a bedside user. The information displayed to the bedside user may include directions and/or prompts for the bedside user to follow. For example, the information may describe what steps to perform next, how to position the robotic drive system 6104, when to deploy the drapes, whether the system is malfunctioning or whether an error is detected, and/or prompt the bedside user to otherwise interact with the system. In some embodiments, the interface 6120 is configured to accept user input to control one or more components, for example, position of the drive table.

The interface 6120 may be in communication with one or more portions of the medical device operation system 6100 (for example, the robotic drive system 6104, the fluidics tower 6102, the disposable devices 6108, etc.). In some embodiments, the interface 6120 may be mechanically coupled to the robotic drive system 6104, be housed separately, or be mechanically coupled to another part of the medical device operation system 6100. The interface 6120 may control the joint setup 6122 of the robotic drive system 6104. For example, the interface 6120 may control the transition processes between a storage position and a deployed position, engaging a priming sequence, or controlling fine motor adjustments for providing minor adjustments to the positions of the interventional hubs. Controlling the joint setup 6122 and motors with the interface 6120 advantageously provides greater precision and setup before an operation by individuals present in the operating room.

The interface 6120 may advantageously provide a backup control mechanism to interact with and provide input to control the medical device operation system 6100, for example, in the event that the control system 6106 is rendered incapable of performing an operation.

The joint setup 6122 may include a plurality of joints and motors for controlling the positioning and movements of the robotic drive system 6104. In some embodiments, the joint setup 6122 may initialize the robotic drive system 6104 into a starting position. The initialization process may include transitioning the robotic drive system 6104 from a storage position to a deployed position and vice versa. For example, the joint setup 6122 may be configured to transition the robotic drive system 6104 from a storage position the robotic drive system 6104 to a deployed position such that at least a portion of the robotic drive system 6104 transitions from a compact state to a position where at least a portion of the robotic drive system 6104 is positioned either over or alongside a patient.

Within the robotic drive system 6104, the drive table 6118 may be mechanically coupled with the interface 6120 and the joint setup 6122. The joint setup 6122 may also be electrically connected to the drive table 6118 and the interface 6120. The robotic drive system 6104 may be configured for the joint setup 6122 to transmit electronic signals and data to the drive table 6118 and the interface 6120. Additionally and/or alternatively, the robotic drive system 6104 may be configured for the joint setup 6122 to receive electronic signals and data from the drive table 6118 and the interface 6120.

The control system 6106 may be a collection of components configured to control and operate the robotic control system described above. In some embodiments, the control system is a control console or is coupled to a control console. The control system 6106 may further include an operator controller 6124, an interface 6126, a monitor 6128, and one or more communication devices 6130. The control system 6106 may be locally positioned or remotely positioned. For example, in some embodiments, the control system 6106 may be located in the operating room with the fluidics tower 6102, the robotic drive system 6104, and the one or more disposable devices 6108. Alternatively, the control system 6106 may be located remotely (e.g., in a control room) as illustrated by line A-A. The control system 6106 may include system electronics including one or more processors and one or more memory components (“memory”). The system electronics may be configured to electrically connect the controller 6124, the interface 6126, the monitor 6128, and the one or more communication devices 6130.

The control system 6106 may include means for connecting to other devices. The control system 6106 may transmit and/or receive electronic signals and/or data wirelessly or over a wired connection. In some embodiments, the control system 6106 may include an ethernet port for connecting the system electronics to a network. In some embodiments, the control system 6106 may include one or more ports for tethering to nearby electronic devices via a wired connection. For example, the control system 6106 may have ports to run cables between the control system 6106 and the fluidics tower 6102 and/or robotic drive system 6104. Alternatively, the control system 6106 may be configured to connect wirelessly to nearby electronic devices. For example, the control system 6106 may include a Bluetooth® module or other network capabilities to transmit data wirelessly.

In some embodiments, the control system 6106 may connect to other devices over a network as described above.

The controller 6124 may be any device configured to enable a surgeon to control portions of the medical device operation system 6100 in the same location as the patient. For example, the controller 6124 may be any of the control mechanisms or controllers described herein. The controller 6124 may enable a user to control portions of the fluidics tower 6102, the interventional devices 6108, and the robotic drive system 6104. For example, the controller 6124 may be configured to move the fluidics tower 6102, the interventional devices 6108, and/or the robotic drive system 6104 to desired positions to perform a procedure on a patient as described herein.

The controller 6124 may be part of the control system 6106 or connected, wirelessly or via a wired connection, to the control system 6106.

The interface 6126 may be configured to display information to the surgeon. The interface 6126 may be the display 204 described above configured to display fluoroscopic images, catheter data, or other patient data. The interface 6126 may be a touchscreen device. The interface 6126 be a graphical user interface.

The monitor 6128 may include one or more electronic displays. The monitor 6128 may be any electronic visual computer display that includes a screen and circuitry configured to interpret electronic signals to display one or more images. The monitor may display the interface 6126. In some embodiments, the monitor 6128 may be configured to display fluoroscopic images, catheter data, or other patient data. Alternatively, the monitor 6128 may be configured to display one or more views of the operating room. For example, the monitor 6128 may be configured to display the working area during a procedure by displaying only the surgical site. In another example, the monitor 6128 may display the entire operating room including the surgical technicians. In another example, the monitor 6128 may display more than one view. Displaying a plurality of views to capture the entire operating room may advantageously enhance communication and understanding between the physician and the technicians and/or assistants located in the operating room thereby increasing the efficiency and safety of procedures.

The one or more communication devices 6130 may be any one or more microphones, one or more cameras, and/or one or more audio output devices.

As shown in FIG. 2B, the fluidics tower 6102, the 6104, the control system 6106, and the one or more disposable devices 6108 are connected to a power source. In some embodiments, the fluidics tower 6102 and the control system 6106 may be directly connected to a power source such as an outlet. In some embodiments, the robotic drive system and the one or more disposable devices 6108 may indirectly connect to a power source. For example, the robotic drive system 6104 and the one or more disposable devices 6108 may receive power from the fluidics tower 6102. In such embodiments, the joint setup 6122 of the robotic drive system 6104 may be electrically connected to the electronics tower 6110 of the fluidics tower and the interventional hubs 6134 of the one or more disposable devices 6108 may be electrically connected to the fluidics system 6112 of the fluidics tower 6102 such that power may be transmitted therebetween.

Furthermore, as shown in FIG. 2B, the fluidics tower 6102, the robotic drive system 6104, the control system 6106, and the one or more disposable devices 6108 may be electrically connected and configured to share electrical signals and/or data. In some embodiments, the fluidics tower 6102 may be electrically connected and configured to share electrical signals and/or data with the robotic drive system 6104, the control system 6106, and the one or more disposable devices 6108. For example, the electronics tower 6110 of the fluidics tower 6102 may be electrically connected with the joint setup 6122 of the robotic drive system 6104 and the control system 6106 while the fluidics system 6112 of the fluidics tower 6102 may be electrically connected with the one or more interventional hubs 6134 of the one or more disposable devices 6108. In some embodiments, the electronics tower 6110 may be electrically connected with the control system 6106 via a network, as shown in FIG. 2B.

The one or more interventional hubs 6134 may include a first interventional hub 6136, a second interventional hub 6138, a third interventional hub 6140, and a fourth interventional hub 6142. In some embodiments, the one or more interventional hubs 6134 may be aligned sequentially such that the first interventional hub 6136 may be positioned at a first end and the fourth interventional hub 6142 may be positioned at a second end opposite the first end. In some embodiments the first end may be a proximal end closest to a patient and the second end may be a distal end furthest from the patient. A sterile tray 6132 may separate the one or more interventional hubs 6134 and corresponding interventional devices from a support table. In some embodiments, the sterile tray 6132 forms a sterile barrier.

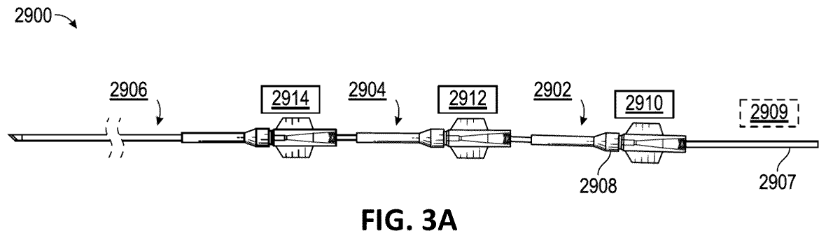

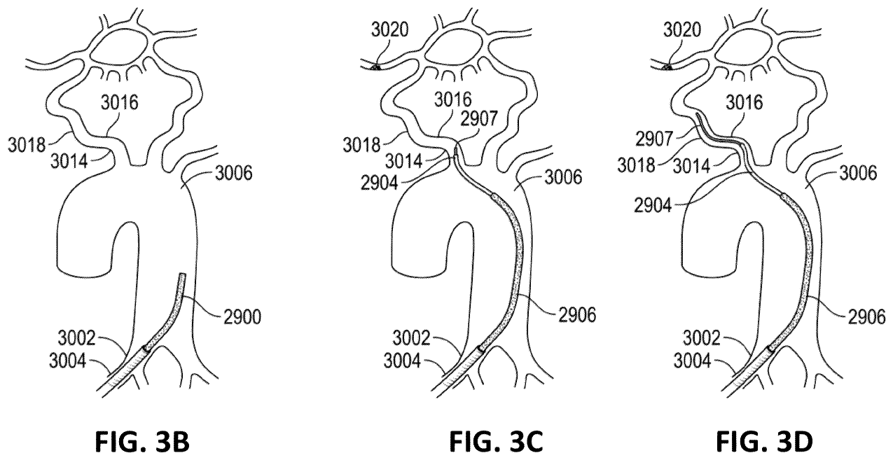

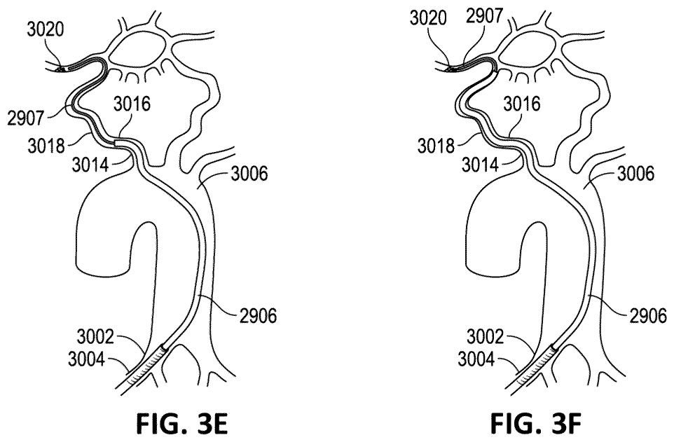

In some embodiments, the first interventional hub 6136 may be a guidewire hub, such as the guidewire hub 126 described above; the second interventional hub 6138 may be a first catheter hub, such as the access catheter hub 2910 described with reference to FIG. 3A; the third interventional hub 6140 may be a second catheter hub configured to engage with and guide a procedure catheter, such as the procedure catheter hub 2912 described with reference to FIG. 3A; and the fourth interventional hub 6142 may be a third catheter hub configured to engage with and guide a guide catheter, such as the guide catheter hub 2914. In some embodiments, the guide catheter may extend distally from the fourth interventional hub 6142.

The fluidics tower 6102 may be electrically connected to the robotic drive system 6104, the control system 6106, and the one or more disposable devices 6108 wherein electrical signals and/or data may be transmitted between therebetween as discussed in greater detail below. The local system may transmit information about the fluidics system 6112, the robotic drive system 6104, and the plurality of interventional devices 6108 to the control system 6106 via the fluidics tower 6102.

The one or more communication devices 6130 may be in electrical communication with a power source. The one or more communication devices 6130 may further be in electrical communication with the electronics tower 6110 and configured to receive and/or transmit electronic signals and/or data therebetween. In some embodiments, the one or more communication devices 6130 is in electrical communication with the electronics tower 6110 via a network. For example, the one or more communication devices 6130 may be electrically coupled to an ethernet cable configured to connect the one or more communication devices 6130 to a network, wherein the electronics tower 6110 may be electrically coupled to the network.

### Imaging System

In some cases, the imaging system 116 can record, capture, and/or transmit fluoroscopic images and/or video of the patient. In some embodiments the images are x-ray fluoroscopic images. The images, as captured by the imaging system 116, can be communicated to the processor 202. With reference to FIG. 1, in some examples, the imaging system 116 can include a display (for example, an imaging display 123) for showing a live fluoroscopic video feed (which includes “native” images) of a patient's vasculature and/or the interventional devices. In such embodiments, the live fluoroscopic video feed of the imaging display 123 may be screen-captured and streamed to the processor 202 (e.g., by software installed on the imaging system 116). Such streaming may be transmitted via the network discussed herein. The imaging system 116 can generate angiograms. In some examples, the imaging system 116 is capable of generating digital subtraction angiograms. In some examples, the imaging system 116 is capable of generating a view where a vessel tree (e.g., a vessel tree generated by digital subtraction angiography) is overlaid onto the native images generated by the imaging system 116.

An imaging display 123 such as for viewing fluoroscopic images, catheter data (e.g., Fiber Bragg grating fiber optics sensor data or other force or shape sensing data) or other patient data may be carried by the support table 120 and or patient support 112. Alternatively, the imaging display 123 may be remote to the patient, such as behind radiation shielding, in a different room from the patient, or in a different facility than the patient. The imaging display 123 may be provided in embodiments where there is a display 204 associated with the processor 202, such that there are multiple displays.

In some embodiments, imaging system 116 be capable of generating images at a frequency of up to 30 Hertz. In other embodiments, the imaging system 116 may be capable of generating images at a frequency of about 4, 5, 7, 10, 15, 20, 25, 30, 35, 40, 45, 50, 55, or 60 Hz, or in a range defined by any two of the preceding values, though in some cases, values outside of those listed herein may be suitable.

In some examples, the guidewire and/or catheter can include components that can readily be discerned via the images. For instance, in some examples using x-ray fluoroscopic images, at least a portion of the guidewire or catheter can be radio-opaque. In some such examples, a tip of the guidewire or the catheter can include a radio-opaque material.

### Catheter Drive System

As discussed, the processor 202 can communicate with the catheter drive system 118. In some embodiments, the catheter drive system 118 may include a drive table positioned over or alongside the patient, and the catheter drive system 118 can axially advance, retract, and in some cases rotate and/or laterally deflect two or three or more different (e.g., concentrically or side by side oriented) intravascular devices. In some embodiments, an interventional device of the catheter drive system 118 may include or be a catheter, a guidewire, and/or a guidewire/catheter stack. The catheter drive system 118 may include one or more hubs. Each hub is moveable along a path along the surface of the drive table to advance or retract the interventional device as desired. Each hub may also contain mechanisms to rotate or deflect the interventional device as desired, and may be connected to fluid delivery tubes of the type conventionally attached to a catheter hub. Each hub can be in electrical communication with the processor 202, which may provide control. The electrical communication between each hub and the processor 202 may be either via hard wired connection, RF wireless connection, a network in accordance with the present disclosure, or a combination thereof.

In some embodiments, each hub can be independently movable across the surface of a sterile field barrier membrane carried by the drive table. Each hub may be releasably magnetically coupled to a unique drive carriage on the table side of the sterile field barrier. The drive system can independently move each hub in a proximal or distal direction across the surface of the barrier, to move the corresponding interventional device proximally or distally within the patient's vasculature.

The carriages on the drive table, which magnetically couple with the hubs to provide linear motion actuation, may be universal. Functionality of the catheters/guidewire are provided based on what is contained in the hub and the shaft designs. This allows flexibility to configure the system to do a wide range of procedures using a wide variety of interventional devices on the same drive table. Additionally, the interventional devices and methods disclosed herein can be readily adapted for use with any of a wide variety of other drive systems (e.g., any of a wide variety of robotic surgery drive systems).

Again with reference to FIG. 1, the catheter drive system 118 may be a robotic drive system. In some embodiments, the catheter drive system 118 may be controllable by the processor 202. The catheter drive system 118 may include a support table 120 for supporting, for example, a guidewire hub 126, an access catheter hub 128 and a guide catheter hub 130. In the present context, the term ‘access’ catheter can be any catheter having a lumen with at least one distally facing or laterally facing distal opening, that may be utilized to aspirate thrombus, provide access for an additional device to be advanced therethrough or therealong, or to inject saline or contrast media or therapeutic agents.

The catheter drive system 118 may include an interventional device including an elongate, flexible body, having a proximal end and a distal end. A hub is provided on the proximal end. Certain embodiments of hub assemblies described herein include a housing for coupling an interventional device thereto, components (e.g., rollers) for directly coupling to and moving along a drive table, and a magnet(s) for magnetically coupling to a hub adapter across a sterile barrier. In various embodiments, a hub assembly can refer to an apparatus having a single assembly that can have a housing, or a hub assembly can generally refer to an apparatus having two (or more) subassemblies (e.g., a first subassembly and a second subassembly) and each subassembly can have a housing. In some embodiments of a hub assembly having two subassemblies, the hub assembly can include a first subassembly (a “hub”) that can be configured to couple to and/or house at least a portion of an interventional device. The first subassembly may be removably attachable to a second subassembly (a “mount”) which is configured to magnetically couple to a hub adapter across a sterile barrier and move along a drive table. In such two-part embodiments, the hub and mount together form the hub assembly. Such hub assemblies may allow for a hub (first subassembly) to be removed from a mount (second subassembly) which can be advantageous, for example, so that a different hub can be coupled to the same mount or so that the hub may be used separately from the mount (e.g., for a manual procedure). An arrangement of a hub assembly having a hub that is releasably couplable to a mount can allow for replacement of a hub with a different hub having a different interventional device coupled thereto without breaking a magnetic connection with a hub adapter. For example, such an arrangement may allow for a hub coupled to an access catheter to be removed from a mount and replaced with a hub coupled to one or more procedure catheters without breaking a magnetic connection between active and passive magnetic sides of the coupling of the hub adapter and hub assembly (e.g., between the hub adapter and the mount). In some embodiments, the mount may be a magnetically driven member, an axially driven member, a puck, a slider, a shuttle, or a stage. The fluidics systems described herein can relate to various embodiments of systems that include a hub assembly having a hub, or a hub and a mount, regardless of whether they are described in reference to a hub, or a hub and mount, unless explicitly indicated or indicated by context.

Any of the guidewire hub, access catheter hub and procedure catheter hub may be further provided with a rotational drive, for rotating the corresponding interventional device with respect to the hub. The hub may be further provided with an axial drive mechanism to distally advance or proximally retract a control element extending axially through the interventional device, to adjust a characteristic such as shape or flexibility of the interventional device. In some embodiments, at least one control element may be an axially movable tubular body or fiber, ribbon, or wire such as a pull wire extending through the interventional device to, for example, a distal deflection zone. In some embodiments, any number of control elements may be advanced, retracted, or otherwise moved in a similar manner.

Again with reference to FIG. 1, a guidewire hub 126 is carried by the support table 120 and is moveable along the table to advance a guidewire into and out of the patient 114. An access catheter hub 128 is also carried by the support table 120 and is movable along the table to advance the access catheter into and out of the patient 114. The access catheter hub 128 may also be configured to rotate the access catheter in response to manipulation of a rotation control, and may also be configured to laterally deflect a deflectable portion of the access catheter, in response to manipulation of a deflection control.

More or fewer interventional device hubs may be provided depending upon the desired clinical procedure. For example, in certain embodiments, a diagnostic angiogram procedure may be performed using only a guidewire hub 126 and an access catheter hub 128 for driving a guidewire and an access catheter (in the form of a diagnostic angiographic catheter), respectively. Multiple interventional devices 122 extend between the support table 120 and (in the illustrated example) a femoral access point 124 on the patient 114. Depending upon the desired procedure, access may be achieved by percutaneous or cut down access to any of a variety of arteries or veins, such as the femoral artery or radial artery. Although disclosed herein primarily in the context of neurovascular access and procedures, the robotic drive system and associated interventional devices can readily be configured for use in a wide variety of additional medical interventions, in the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, intra-cardiac or structural heart applications, such as valve repair or replacement, or in in any endoluminal procedures).

In the illustrated example, a guidewire hub 126 is carried by the support table 20 and is moveable along the table to advance a guidewire into and out of the patient 114. An access catheter hub 28 is also carried by the support table 120 and is movable along the table to advance the access catheter into and out of the patient 114. The access catheter hub may also be configured to rotate the access catheter in response to manipulation of a rotation control, and may also be configured to laterally deflect a deflectable portion of the access catheter, in response to manipulation of a deflection control.

The catheter drive system 118 may include sensors capable of measuring movement and/or changes in position of an associated interventional devices (e.g., a catheter and/or a guidewire), for example at a hub such as the guidewire hub 126, the access catheter hub 128, and/or the guide catheter hub 130. Additionally or alternatively, the catheter drive system 118 may include sensors capable of measuring force exerted by or on the interventional device. The catheter drive system 118 can transmit such movement data to the processor 202. Examples of such sensors capable of producing drive system sensor data are discussed herein.

In some implementations, a number of deflection sensors may be placed along a catheter length to identify buckling. Identifying buckling may be performed by sensing that a hub is advancing distally, while the distal tip of the catheter or interventional device has not moved. In some implementations, the buckling may be detected by sensing that an energy load (e.g., due to friction) has occurred between catheter shafts. Such deflection sensors may be capable of transmitting data to a processor 202 in accordance with the present disclosure. The data of the deflection sensors may be used the processor 202 to help detect catheter prolapse.

The catheter drive system 118 may include a procedure catheter hub configured to manipulate, among other catheters, one or more procedure catheters. Following robotic placement of the guidewire, access catheter and guide catheter (which may also be referred to as an “insert catheter”) such that the guide catheter achieves access to the intended blood vessel, the guidewire and access catheter may be proximally withdrawn and the one or more procedure catheters advanced through and beyond the guide catheter, with or without guidewire support (said guidewire may be smaller in diameter and/or more flexible than the guidewire used to gain supra aortic access), to reach a more distal neurovascular treatment site. The one or more procedure catheters may include one or more of each of: an aspiration catheter, an embolic deployment catheter, a stent deployment catheter, a flow diverter deployment catheter, an access catheter, a diagnostic angiographic catheter, an imaging catheter, a physiological sensing/measuring catheter, an infusion or injection catheter, an ablation catheter, an RF ablation catheter or guidewire, a balloon catheter, or a microcatheter used to deliver a stent retriever, a balloon catheter, or a stent retriever.

Any of the hubs disclosed herein may further include a fluid injection port and/or a wireless RF transceiver for communications and/or power transfer. In some embodiments, contrast media detectable by the imaging system 116 may be injected via the fluid injection port.

Any of the hubs disclosed herein may further include a sensor for detecting a parameter of interest. The sensor, in some instances, may be positioned on a flexible body. The sensor may include a pressure sensor or an optical sensor. In some embodiments, the sensor may include one or more of a force sensor, a positioning sensor, a temperature sensor, and/or an oxygen sensor. In some embodiments, the sensor may include a Fiber Bragg grating (FBG) sensor. For example, a FBG sensor (e.g., an optical fiber) may detect strain locally that can facilitate the detection and/or determination of force being applied. The device may further include a plurality of sensors. The plurality of sensors may each include one or more of any type of sensor disclosed herein. In some embodiments, a plurality (e.g., 3 or more) of sensors (e.g., FBG sensors) may be distributed around a perimeter to facilitate the detection and/or determination of shape. The position of the device, in some instance, may be determined through the use of one or more sensors to detect and/or determine the position. For example, one or more optical encoders may be located in or proximate to one or more the motors that drive linear motion such that the optical encoders may determine a position.

In some embodiments, as discussed herein, each hub may be releasably magnetically coupled to a unique drive carriage. It may be desirable to measure elastic forces across the magnetic coupling between the hub and corresponding carriage, using the natural springiness (compliance) of the magnetic coupling to measure the force applied to the hub. As depicted in FIGS. 4A and 4B, the magnetic coupling between a hub and a carriage creates a spring. When a force is applied to the hub, the hub will move a small amount relative to the carriage. See FIG. 4A. In robotics, this is referred to as “a series elastic actuator.” This property can be used to measure the force applied from the carriage to the hub. To measure the force, the relative distance between the hub and the carriage is determined and characterize some effective spring constant k between the two components. See FIG. 4B.

The relative distance between the hub and the carriage can be measured in multiple different ways. In some embodiments, the relative distance between the hub and carriage is measured by a magnetic sensor (e.g., a Hall effect sensor between hub and carriage). A magnet can be mounted to either the hub or carriage, and a corresponding magnetic sensor is mounted on the other device (carriage or hub). The magnetic sensor can be a Hall effect sensor, a magnetoresistive sensor, or another type of magnetic field sensor. Generally, multiple sensors may be used to increase the reliability of the measurement. This reduces noise and reduces interference from external magnetic fields.

Other non-contact distance sensors can also be used. These include optical sensors, inductance sensors, and capacitance sensors. Optical sensors may be configured in a manner that avoids accumulation of blood or other fluid in the interface between the hubs and carriages. In some implementations, wireless (i.e., inductive) power may be used to translate movement and/or transfer information across the sterile barrier between a drive carriage and a hub, for example.

The magnetic coupling between the hub and the carriage has a shear or axial breakaway threshold. In some embodiments, the breakaway threshold may be 50, 100, 200, 400, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 grams or within a range defined by any two of the previous values, though in some cases other ranges or values may be suitable. In some embodiments, the breakaway threshold may be about 400 grams. In some embodiments, the breakaway threshold may be 1000 grams. In some embodiments, the breakaway threshold may be 1000 grams or more. The processor can be configured to compare the axial force applied to the catheter to a preset axial trigger force which if applied to the catheter is perceived to create a risk to the patient. If the trigger force is reached, the processor may be configured to generate a response such as a visual, auditory or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and or torque in the vicinity of the sensor.

It may also be desirable to understand the three-dimensional configuration of the catheter or guidewire during and/or following transvascular placement. Shape sensing fiber optics such as an array of FBG fibers to sense the shape of catheters and guidewires. By using multiple force sensing fibers that are a known distance from each other, the shape along the length of the catheter/guidewire can be determined.

A resistive strain gauge may be integrated into the body of the catheter or guidewire to measure force or torque. Such as at the distal tip and/or proximal end of the device.

Measurements of force and/or torque applied to the catheter or guidewire shafts can be used to determine applied force and/or torque above a safety threshold. When an applied force and/or torque exceeds a safety threshold, a warning may be provided to a user (e.g., via the display 204). Applied force and/or torque measurements may also be used to provide feedback related to better catheter manipulation and control. Applied force and/or torque measurements may also be used (e.g., by the processor 202) with processed fluoroscopic imaging information to determine or characterize distal tip motion.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the carriages. Each hub may have at least one magnet attached to it. The robotic table would have a linear array of corresponding magnetic sensors going the entire length of the table. A processor can be configured to determine the location of the magnet along the length of the linear sensor array, and display axial position information to the physician.

The foregoing may alternatively be accomplished using a non-contact inductive sensor to directly measure the position of the hub through the sterile barrier. Each hub or carriage may be provided with an inductive “target” in it. The robotic table may be provided with an inductive sensing array over the entire working length of the table. As a further alternative, an absolute linear encoder may be used to directly measure the linear position of the hubs or carriages. The encoder could use any of a variety of different technologies, including optical, magnetic, inductive, and capacitive methods.

In one implementation, a passive (no electrical connections) target coil may be carried by each hub. A linear printed circuit board (PCB) may run the entire working length of the table (e.g., at least about 1.5 meters to about 1.9 meters) configured to ping an interrogator signal which stimulates a return signal from the passive coil. The PCB is configured to identify the return signal and its location.

Axial position of the carriages may be determined using a multi-turn rotary encoder to measure the rotational position of the pulley, which directly correlates to the linear position of the carriage. Direct measurement of the location of the carriage may alternatively be accomplished by recording the number of steps commanded to the stepper motor to measure the rotational position of the pulley, which directly correlates to the linear position of the carriage.

Proximal torque applied to the catheter or guidewire shaft may be determined using a dual encoder torque sensor. With reference to FIG. 4C, a first encoder 444 and a second encoder 446 may be spaced axially apart along the shaft 448, for measuring the difference in angle over a length of flexible catheter/tube. The difference in angle is interpolated as a torque, since the catheter/tube has a known torsional stiffness. As torque is applied to the shaft, the slightly flexible portion of the shaft will twist. The difference between the angles measured by the encoders (dθ) tells us the torque. T=k*dθ, where k is the torsional stiffness.

Though certain drive systems are discussed herein, other drive systems may be suitably implemented. Drive systems may allow for control of interventional devices and sensing of position, motion, force, strain, etc., or a combination thereof of the interventional devices.

Additional details regarding drive systems can be found in U.S. application Ser. No. 19/228,455, entitled DRIVE TABLE, filed Jun. 4, 2025, and U.S. application Ser. No. 19/228,468, METHOD FOR ROBOTICALLY CONTROLLING INTERVENTIONAL DEVICE ASSEMBLY, filed Jun. 4, 2025, each of which are incorporated by reference in their entireties.

### Multi Catheter Stack and Supra-Aortic Access

In a manual catheter procedure, a physician often stands to a patient's right side and inserts interventional devices from the physician's right to the physician's left when facing the patient. Certain embodiments of robotic control mechanisms described herein may be configured to mimic the movements a physician makes in a manual catheter procedure. For example, certain embodiments of robotic control mechanisms described herein include controls that are operated by left/right motion from the perspective of a user (e.g., a physician) operating the control to command insertion/withdrawal of an interventional device. Certain embodiments of robotic control mechanisms described herein include controls that are operated by rolling or rotational motion from the perspective of a user (e.g., a physician) operating the control to command roll or rotation of an interventional device.

FIG. 3A illustrates a side elevational schematic view of a multi catheter interventional device assembly 2900 for combined supra-aortic access and/or neurovascular site access and procedure (e.g., aspiration), as described herein. The multi catheter assembly 2900 may be configured for a robotic procedure. For instance, the catheter assembly 2900 may be controlled by a drive system in accordance with the present disclosure.

The interventional device assembly 2900 includes an insert or access catheter 2902, a procedure catheter 2904, and a guide catheter 2906. Other components are possible including, but not limited to, one or more guidewires (e.g., optional guidewire 2907), one or more guide catheters, an access sheath and/or one or more other procedure catheters and/or associated catheter (control) hubs. In some embodiments, the assembly 2900 may also be configured with an optional deflection control 2908 for controlling deflection of one or more catheters of assembly 2900.

In operation, the multi-catheter assembly 2900 may be used without having to exchange hub components. For example, in the two stage procedure disclosed previously, a first stage for achieving supra-aortic access, includes mounting an access catheter, guide catheter and guidewire to the support table. Upon gaining supra aortic access, the access catheter and guidewire were typically removed from the guide catheter. Then, a second catheter assembly is introduced through the guide catheter after attaching a new guidewire hub and a procedure catheter hub to the corresponding drive carriage on the support table.

The devices of the multi catheter assembly 2900 may be operated using a control mechanism (e.g., a handheld controller, a user interface, etc.) as described in further detail herein. The control mechanism may be operated to cause independent or simultaneous axial translation of the interventional devices of the multi catheter assembly 2900 (e.g., along a drive surface). The control mechanism may be operated to cause independent or simultaneous rotational movement of one or more of the interventional devices of the multi catheter assembly 2900.

The single multi catheter assembly 2900 of FIG. 3A is configured to be operated without having to remove hubs and catheters and without the addition of additional assemblies and/or hubs. Thus, the multicomponent access and procedure configuration of assembly 2900 may utilize a guidewire 2907 manufactured to function as an access guidewire and a navigation guidewire to allow for sufficient access and support, and navigation to the particular distal treatment site. In a non-limiting example configured for robotic implementation, a catheter assembly may include a guidewire hub (e.g., guidewire hub 2909 or guidewire hub 26 positioned on a drive table and to the right of catheter 2902), an insert or access catheter hub 2910, a procedure catheter hub 2912, a guide catheter hub 2914 and corresponding catheters. In certain embodiments, one or more of the hubs may include or be coupled to a hemostasis valve (e.g., a rotating hemostasis valve) to accommodate introduction of interventional devices therethrough. In some embodiments, any of the control mechanisms described herein can include at least one control for opening and closing a hemostasis valve.

Additional details regarding hemostasis valves are included in U.S. patent application Ser. No. 17/879,614, entitled MULTI CATHETER SYSTEM WITH INTEGRATED FLUIDICS MANAGEMENT, filed Aug. 2, 2022, which is hereby expressly incorporated by reference in its entirety herein.

One or more of interventional device and hub combinations may further include fluidics connections for coupling to fluid sources and/or vacuum sources. For example, each of the insert or access catheter 2902, the procedure catheter 2904, and the guide catheter 2906 may be in fluid communication with a saline source, a contrast source, and/or a vacuum source. In some embodiments, any of the control mechanisms described herein can include at least one control for initiating and/or terminating the introduction of fluids to one or more of the catheters and/or aspiration of fluids from one or more of the catheters. For example, any of the control mechanisms described herein can include at least one control for opening and/or closing one or more valves to initiate the introduction of fluids to one or more of the catheters and/or aspiration of fluids from one or more of the catheters. For example, any of the control mechanisms described herein can be used to control various components (e.g., manifold valves, pumps, hemostatic valves, hubs, and/or catheters) of a fluidics systems as described in U.S. patent application Ser. No. 17/879,614, entitled Multi Catheter System With Integrated Fluidics Management, filed Aug. 2, 2022, the entirety of which is hereby incorporated by reference herein.

In some embodiments, the control mechanisms described herein may allow a user to simultaneously control movement of a catheter (e.g., axial and/or rotational movement) and a fluidics system (e.g., for introduction of fluids and/or aspiration).

In some implementations, other control operations beyond translational movement and rotational movement may be carried out using any of the controls described herein. For example, the controls may be configured to drive a shape change and/or stiffness change of a corresponding interventional device. Controls may be toggled between different operating modes. For example, controls may be toggled between movement driven by acceleration and velocity to movement that reflects actual linear displacement or rotation.

In some implementations, the control mechanisms may be provided with a visual display or other indicator of the relative positions of the controls which may correspond the relative positions of the interventional devices. Such displays may depict any or all movement directions, instructions, percentage of movements performed, and/or hub and/or catheter indicators to indicate which device is controlled by a particular control. In some implementations, the display may depict applied force or resistance encountered by the catheter or other measurement being detected or observed by a particular hub or interventional component.

The systems described herein may compare an actual fluoroscopic image position to an input displacement from the controller. A static fluoroscopic image of the patient may be captured in which the patient's vasculature is indexed relative to bony landmarks or one or more implanted soft tissue fiducial markers. Then a real time fluoroscopic image may be displayed as an overlay, aligned with the static image by registration of the fiducial markers. Visual observation of conformance of the real time movement with the static image, assisted by detected force data can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be beneficial when the shaft buckling, prolapse, kinking, or similar outcome occurs outside of a current fluoroscopic view.

Additional details regarding a controller for a robotic surgical system can be found in U.S. patent application Ser. No. 18/784,630, entitled SYSTEM FOR REMOTE MEDICAL PROCEDURE, filed Jul. 25, 2024, the entirety of which is hereby incorporated by reference herein.

Once access above the aortic arch has been achieved, the insert or access catheter 2902 (associated with insert catheter hub 2910) may be parked in the vicinity of a carotid artery ostia and the remainder or a subset of the catheter assembly may be guided more distally toward a particular site (e.g., a clot site, a surgical site, a procedure site, etc.).

In some embodiments, other smaller procedure catheters may also be added and used at the site. As used herein for catheter assembly 2900, in a robotic configuration of assembly 2900, the catheter 2906 may function as a guide catheter. The catheter 2904 may function as a procedure (e.g., aspiration) catheter. In some embodiments, the catheter 2906 may function to perform aspiration in addition to functioning as a guide catheter, either instead of or in addition to the catheter 2904. The access catheter 2902 may have a distal deflection zone and can function to access a desired ostium. One of skill in the art will appreciate from FIGS. 3B-3F that robotic manipulation of the multi catheter stack are contemplated herein.

In some embodiments, the catheter assembly 2900 (or other combined catheter assemblies described herein) may be driven as a unit to a location. However, each catheter (or guidewire) component may instead be operated and driven independent of one another to the same or different locations.

In a non-limiting example, the catheter assembly 2900 may be used for a diagnostic angiogram procedure. In some embodiments, the assembly 2900 may include only the guidewire 2907 and access catheter 2902 (in the form of a diagnostic angiographic catheter) for performing the diagnostic angiogram procedure or only the guidewire 2907 and the access catheter 2902 may be utilized during the procedure. Alternatively, the guide catheter 2906 and procedure catheter 2904 may be retracted proximally to expose the distal end of the access catheter 2902 (e.g., a few centimeters of the distal end of the access catheter) to perform the diagnostic angiography.

As shown in FIG. 3A, the guide catheter 2906, procedure catheter 2904, access catheter 2902, and guidewire 2907 can be arranged concentrically. In certain embodiments, the guide catheter 2906 may be a ‘large bore’ guide catheter or access catheter having an inner diameter of at least about 0.075 or at least about 0.080 inches in diameter. The procedure catheter 2904 may be an aspiration catheter having an inner diameter within the range of from about 0.060 to about 0.075 inches. The access catheter 2902 may be a steerable catheter with a deflectable distal tip, having an inner diameter within the range of from about 0.025 to about 0.050 inches. The guidewire 2907 may have an outer diameter within the range of from about 0.014 to about 0.020 inches. In one example, the guide catheter 2906 may have an inner diameter of about 0.088 inches, the procedure catheter 2904 about 0.071 inches, the access catheter 2902 about 0.035 inches, and the guidewire 2907 may have an outer diameter of about 0.018 inches.

FIGS. 3B-3F depict an example sequence of steps of introducing a multi-catheter assembly configured to achieve access all the way to the clot, either manually or robotically. FIGS. 3B-3F may be described using the interventional device assembly of FIG. 3A. Other combinations of catheters may be substituted for the interventional device assembly, as will be appreciated by those of skill in the art in view of the disclosure herein.

Referring to FIG. 3B, the three catheter interventional device assembly 2900 is shown driven through an introducer sheath 3002, up through the iliac artery 3004 and into the descending aorta. Next, the access catheter 2902, the procedure catheter 2904 (e.g., 0.071 inch) and the guide catheter 2906 (e.g., 0.088 inch) are tracked up to the aortic arch 3006, as shown in FIG. 3C. Here, the distal end of the guide catheter 2906 may be parked below the aortic arch 3006 and the procedure catheter 2904, access catheter 2902 (positioned within the procedure catheter 2904 and not visible in FIG. 3C), and a guidewire 2907 can be driven into the ostium (e.g., simultaneously or separately). In some embodiments, the access catheter 2902 is advanced out of the procedure catheter 2904 and the guide catheter 2906 to engage the ostium first. After the distal end of the access catheter 2902 is positioned within the desired ostium, the guidewire 2907 can be advanced distally into the ostium to secure access. After the access catheter 2902 and guidewire 2907 are positioned within the desired ostium, the procedure catheter 2904 and/or guide catheter 2906 can be advanced into the ostium (and, in some embodiments, beyond), while using the support of the access catheter 2902 and/or guidewire 2907 to maneuver through the aorta and into the ostium. In the embodiment shown in FIG. 3C, the procedure catheter 2904 has been advanced into the ostium while the guide catheter 2906 has remained parked below the aortic arch 3006.

Referring to FIG. 3D, the guidewire 2907 may be distally advanced and the radiopacity of the guidewire 2907 may be used to confirm under fluoroscopic imaging that access through the desired ostia has been attained. The guidewire 2907 engages the origin of the brachiocephalic artery 3014. The guidewire 2907 is then advanced up to the petrous segment 3018 of the internal carotid artery 3016.

Referring to FIG. 3E, the guide catheter 2906 and the procedure catheter 2904 (positioned within the guide catheter 2906 and not visible in FIG. 3E) are both advanced (e.g., simultaneously or sequentially) over the guidewire 2907 and over the insert or access catheter 2902 (positioned within the procedure catheter 2904 and not visible in FIG. 3E) while the access catheter 2902 remains at the ostium for support. The guidewire 2907 may be further advanced past the petrous segment 3018 to the site of the clot 3020, such as the M1 segment.

Referring to FIG. 3F, the guide catheter 2906 and the procedure catheter 2904 (positioned within the guide catheter 2906 and not visible in FIG. 3F) are advanced (e.g., simultaneously or sequentially) to position the distal tip of the procedure catheter 2904 at the procedure site, for example on the face of the clot 3020. The guidewire 2907 and access catheter 2902 (positioned within the procedure catheter 2904 and not visible in FIG. 3F) are removed, and aspiration of the clot 3020 commences through the procedure catheter 2904. That is, the guidewire 2907 and the access catheter 2902 are proximally retracted to allow aspiration through the procedure catheter 2904. After aspiration of the clot, the procedure catheter 2904 and guide catheter 2906 can be removed (e.g., simultaneously or sequentially). For example, in some embodiments, the procedure catheter 2904 may be removed before removing the guide catheter 2906.

In some embodiments, aspiration may be performed through two catheters (e.g., the procedure catheter 2904 and the guide catheter 2906) simultaneously. For example, during a thrombectomy procedure, the clot 3020 may become engaged with or corked at a distal end of the procedure catheter 2904 (or another inner catheter). In such cases, it may be necessary to remove the procedure catheter 2904 (or other inner catheter) from the vasculature of the patient to remove the clot 3020. As the procedure catheter 2904 (or other inner catheter) is retracted from the guide catheter 2906 (or other outer catheter), debris from the stuck clot 3020 can dislodge. In such embodiments, application of vacuum at both the procedure catheter 2904 (or other inner catheter) and the guide catheter 2906 (or other outer catheter) can beneficially prevent the debris from flowing distally into the vasculature of the patient by aspirating the debris and thus reducing the risk of embolization.

In some embodiments, the clot 3020 may become engaged with or corked at the distal end of the guide catheter 2906 (or other outer catheter). Vacuum through the guide catheter 2906 (or other outer catheter) may prevent dislodgement of the clot 3020 from the guide catheter 2906 (or other outer catheter). In some instances, it may not be readily apparent if portions of a clot are engaged with the guide catheter 2906 (or other outer catheter) or the procedure catheter 2904 (or other inner catheter). In such instances, vacuum through both the guide catheter 2906 (or other outer catheter) and the procedure catheter 2904 (or other inner catheter) may prevent debris from flowing distally into the vasculature of the patient by aspirating the debris and thus reducing the risk of embolization.

The catheter assembly 2900 may be used to perform a neurovascular procedure, as described in FIGS. 3B-3F. For example, the neurovascular procedure may be a neurovascular thrombectomy. The steps of the procedure may include providing an assembly that includes at least a guidewire, an access catheter, a guide catheter, and a procedure catheter. For example, the catheter assembly 2900 includes a guidewire 2907, an access (e.g., insert) catheter 2902, a guide catheter 2906, and at least one procedure catheter 2904. The procedure catheter 2904 may include an aspiration catheter, an embolic deployment catheter, a stent deployment catheter, a flow diverter deployment catheter, a diagnostic angiographic catheter, a stent retriever catheter, a clot retriever catheter, a balloon catheter, a catheter to facilitate percutaneous valve repair or replacement, an ablation catheter, and/or an RF ablation catheter or guidewire.

The neurovascular procedure may further include steps of coupling the assembly to a non-robotic or a robotic drive system, and driving the assembly to achieve supra-aortic access. The steps may further include driving a subset of the assembly to a neurovascular site, and performing the neurovascular procedure using a subset of the assembly. The subset of the assembly may include the guidewire, the guide catheter, and the procedure catheter.

Each of the guidewire 2907, the access catheter 2902, the guide catheter 2906, and the procedure catheter 2904 is configured to be adjusted by a respective hub. For example, the guidewire 2907 may include (or be coupled to) a hub installed on one of the tray assemblies described herein. Similarly, the access catheter 2902 may be coupled to catheter hub 2910. The guide catheter 2906 may be coupled to the guide catheter hub 2914. The procedure catheter 2904 may be coupled to the procedure catheter hub 2912.

In general coupling of the assembly may include magnetically coupling a first hub 2909 on the guidewire 2907 to a first drive magnet, magnetically coupling a second hub 2910 on the access catheter 2902 to a second drive magnet, magnetically coupling a third hub 2912 on the procedure catheter 2904 to a third drive magnet, and magnetically coupling a fourth hub 2914 on the guide catheter 2906 to a fourth drive magnet. In general, the first drive magnet, the second drive magnet, the third drive magnet, and the fourth drive magnet are each independently movably carried by a drive table, as described with respect to tray assemblies and controls described herein. In some embodiments, the first drive magnet, the second drive magnet, the third drive magnet, and the fourth drive magnet are coupled (e.g., to their respective catheter hubs) through a sterile barrier (e.g., a sterile and fluid barrier) and independently movably carried by a drive table having a plurality of driven magnets. In some embodiments, two or more drive magnets can be tethered or otherwise coupled together to move as a unit in response to commands from a single controller tethered or otherwise coupled to one of the drive magnets.

In some implementations, the steps of performing the neurovascular procedure may include driving the assembly in response to movement of each of the hub adapters along a support table until the assembly is positioned to achieve supra-aortic vessel access. The hub adapters may include, for example, a coupler/carriage that acts as a shuttle by advancing proximally or distally along a track in response to operator instructions. The hub adapters described herein may each include at least one drive magnet configured to couple with a driven magnet carried by the respective hub. This provides a magnetic coupling between the drive magnet and driven magnet through the sterile barrier such that the respective hub is moved across the top of the sterile barrier in response to movement of the hub adapter outside of the sterile field. Movement of the hub adapter is driven by a drive system carried by the support table in which the guidewire hub 2909, the guide catheter hub 2914, the procedure catheter hub 2912, and the access catheter hub 2910 are installed upon.

The steps may further include driving a subset of the assembly in response to movement of each of the hub adapters along the support table until the subset of the assembly is positioned to perform a neurovascular procedure at a neurovascular treatment site. The subset of the assembly may include the guidewire 2907, the guide catheter 2906, and the procedure catheter 2904.

In some embodiments, the guidewire 2907, the guide catheter 2906 and the procedure catheter 2904 are advanced as a unit through (with respect to the guidewire 2907) and over (with respect to the guide catheter 2906 and the procedure catheter 2904) at least a portion of a length of the access (e.g., insert) catheter 2902 after supra-aortic access is achieved.

In some embodiments, the catheter assembly 2900 may be part of a robotic control system for achieving supra-aortic access and neurovascular treatment site access, as described in FIGS. 3B-3F. In some embodiments, the catheter assembly 2900 may be part of a manual control system for achieving supra-aortic access and neurovascular treatment site access. In some embodiments, the catheter assembly 2900 may be part of a hybrid control system (with manual and robotic components) for achieving supra-aortic access and neurovascular treatment site access. For example, in such hybrid systems, supra-aortic access may be robotically driven while neurovascular site access and embolectomy or other procedures may be manual. Alternatively, in such hybrid systems, supra-aortic access may be manual while neurovascular site access may be robotically achieved. Still further, in such hybrid systems, any one or more of: the guidewire, access catheter, guide catheter, or procedure catheter may be robotically driven or manually manipulated.

An example robotic control system may include at least a guidewire hub (e.g., guidewire hub 2909) configured to adjust each of an axial position and a rotational position of a guidewire 2907. The robotic control system may also include an access catheter hub 2910 configured to adjust axial and rotational movement of an access catheter 2902. The robotic control system may also include a guide catheter hub 2914 configured to control axial movement of a guide catheter 2906. The robotic control system may also include a procedure catheter hub 2912 configured to adjust an axial position and a rotational position of a procedure catheter 2904.

In some embodiments, the procedure catheter hub 2912 is further configured to laterally deflect a distal deflection zone of the procedure catheter 2904.

In some embodiments, the guidewire hub 2909 is configured to couple to a guidewire hub adapter by magnetically coupling the guidewire hub to a first drive magnet. The access catheter hub 2910 is configured to couple to an access catheter hub adapter by magnetically coupling the access catheter hub 2910 to a second drive magnet. The procedure catheter hub 2912 is configured to couple to a procedure catheter hub adapter by magnetically coupling the procedure catheter hub 2912 to a third drive magnet. The guide catheter hub 2914 is configured to couple to a guide catheter hub adapter by magnetically coupling the guide catheter hub 2914 to a fourth drive magnet. In some embodiments, the first drive magnet, the second drive magnet, the third drive magnet, and the fourth drive magnet are independently movably carried by a drive table.

In some embodiments, the robotic control system includes
a first driven magnet on the guidewire hub 2909. The first driven magnet may be configured to cooperate with the first drive magnet such that the first driven magnet is configured to move in response to movement of the first drive magnet. In some embodiments, the first drive magnet is configured to move outside of a sterile field separated from the first driven magnet by a barrier while the first driven magnet is within the sterile field. In some embodiments, a position of the first driven magnet is movable in response to manipulation of a procedure drive control on a control console associated with the drive table. Drive magnets and driven magnet interactions are described in detail with respect to FIGS. 4A and 4B.

In some embodiments, the robotic control system includes a second driven magnet on the access catheter hub 2910. The second driven magnet may be configured to cooperate with the second drive magnet such that the second driven magnet is configured to move in response to movement of the second drive magnet. In some embodiments, the second drive magnet is configured to move outside of a sterile field separated from the second driven magnet by a barrier while the second driven magnet is within the sterile field.

In some embodiments, the robotic control system includes a third driven magnet on the procedure catheter hub 2912. The third driven magnet may be configured to cooperate with the third drive magnet such that the third driven magnet is configured to move in response to movement of the third drive magnet. In some embodiments, the third drive magnet is configured to move outside of a sterile field separated from the third driven magnet by a barrier while the third driven magnet is within the sterile field.

In some embodiments, the robotic control system includes a fourth driven magnet on the guide catheter hub 2914. The fourth driven magnet may be configured to cooperate with the fourth drive magnet such that the fourth driven magnet is configured to move in response to movement of the fourth drive magnet. In some embodiments, the fourth drive magnet is configured to move outside of a sterile field separated from the fourth driven magnet by a barrier while the fourth driven magnet is within the sterile field. In some embodiments, there may be more than four driven magnets and corresponding catheter hubs for control of additional catheters.

In some embodiments, devices (e.g., hubs, hub adapters, interventional devices, and/or trays) described herein may be used during a robotically driven procedure. For example, in a robotically driven procedure, one or more of the interventional devices may be driven through vasculature and to a procedure site. Robotically driving such devices may include engaging electromechanical components that are controlled by user input. In some implementations, users may provide the input at a control system that interfaces with one or more hubs and hub adapters.

In some embodiments, the hubs, hub adapters, interventional devices, and trays described herein may be used during a non-robotic (e.g., manually driven) procedure. Manually driving such devices may include engaging manually with the hubs to affect movement of the interventional devices.

In some embodiments, the devices described herein may be used to carry out a method of performing an intracranial procedure at an intracranial site. The method of performing the intracranial procedure may include any of the same steps as described herein for performing a neurovascular procedure. The procedure may be robotically performed, manually performed, or a hybridized combination of both.

While the foregoing describes magnetic coupling of hubs to drive magnets, in other embodiments, any of the interventional devices and/or hubs may be mechanically coupled to a drive system. Any of the methods described herein may include steps of mechanically coupling one or more interventional devices (e.g., the guidewire 2907, the access catheter 2902, the procedure catheter 2904, and/or the guide catheter 2906) and/or one or more hubs (e.g., the guidewire hub 2909, the access catheter hub 2910, the procedure catheter hub 2912, and/or the guide catheter hub 2914) with one or more drive mechanisms.

### Processor

The processor 202 can process the images of the imaging system 116 and transmit processed images and/or native images to the display 204. In some cases, the display 204 can mirror the image feed shown on the imaging display 123 of the imaging system 116 and show it on a portion of the display 204. In some examples, the display 204 can show the live image feed (i.e., mirroring the imaging display 123) alongside images processed by the processor 202. The display 204 can beneficially allow clinicians to visualize the one or more interventional devices in the vasculature of a patient on the display 204 as the interventional devices are advanced in and/or retracted from the body of the patient. The processor 202 can communicate to the imaging system 116 and the catheter drive system 118 as discussed herein, for example over a network. It is to be understood that the processor 202 may include any suitable number of processors and/or virtual machines in communication with each other and that the processor 202 need not be a single processor.

The processor 202 and/or the display 204 may be local or remote to the patient, the imaging system 116, and catheter drive system 118. In examples where either or both of the processor 202 and the display 204 are remote to the patient, the imaging system 116, and the catheter drive system 118, the processor 202 and/or the display 204 may be positioned behind radiation shielding, in a different room from the patient, or in a different facility than the patient.

The processor 202 can compare a position of the guidewire/catheter stack as perceived from images of the imaging to drive system sensor data. In some examples, a static image of the patient may be captured in which the patient's vasculature is indexed relative to bony landmarks or one or more implanted soft tissue fiducial markers. Then a real-time image may be displayed as an overlay, aligned with the static image by registration of the fiducial markers. Visual observation of conformance of the real time movement with the static image, assisted by detected force data (e.g., from the catheter drive system) can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be particularly beneficial when the shaft buckling, prolapse, kinking, or similar outcome occurs outside of a current fluoroscopic view.

The location of the catheters and guidewires within a patient's anatomy may also be determined by the processor 202. The processor 202 may be capable of processing the fluoroscopic image using machine vision, such as to determine the distal tip position, distal tip orientation, and/or guidewire shape. Comparing distal tip position or movement or lack thereof as determined based on fluoroscopic images generated by the imaging system 116 to commanded or actual proximal catheter or guidewire movement measured by the catheter drive system 118, may be used to detect a loss of relative motion. Such relative loss of motion may be indicative of a device shaft buckling, prolapse, kinking, or a similar outcome. Such buckling, prolapse, kinking, or the like may be detected, for example, along the catheter shaft length inside the body (e.g., in the aorta) or outside the body between hubs of the catheter drive system 118.

The processor 202 may be capable of processing images in real time to provide position/orientation data at up to 30 Hertz. In some embodiments, the processor 202 can process images at the same rate that the imaging system 116 generates images. In some embodiments, the processor 202 may be capable of processing images in real time to provide position/orientation data at up to 4, 5, 7, 10, 15, 20, 25, 30, 35, 40, 45, 50, 55, 60, 65, 70, 75, 80, 85, 90, 95, 100, 105, 110, 115, or 120 Hz, or in a range defined by any two of the preceding values, though in some cases, values outside of those listed herein may be suitable. This technique can provide data while the imaging system 116 is active and generating fluoroscopic images.

In some embodiments, machine vision algorithms of the processor 202 can be used to generate and suggest optimal catheter manipulations to access or reach anatomical landmarks, similar to driver assist. The machine vision algorithms may utilize data to automatically drive the catheters depending on the anatomy presented by fluoroscopy.

In some embodiments, the processor 202 may be capable of fluidic control. For example, the processor 202 may be capable of controlling components of a fluidics system local to the patient. In such examples, the processor 202 may be capable of initiating and/or terminating the introduction of fluids to a catheter (e.g., saline, contrast media, etc.) and/or to initiate and/or terminate aspiration of fluids from a catheter.

The processor 202 may be capable of carrying out certain steps of any of the methods discussed herein, for example steps of processing images, determining catheter position, detecting catheter prolapse, predicting catheter prolapse, and/or notifying a user.

In some implementations, the processor 202 may be in communication with or integrated with a control system 206 configured to transmit movement commands to the catheter drive system 118. In some examples, the control system 206 may be a robotic control system. Such a control system may allow a healthcare practitioner to control movement of the catheter drive system 118 remotely. Controllers and control systems are discussed in U.S. application Ser. No. 18/525,267, entitled METHOD FOR ROBOTICALLY CONTROLLING SUBSETS OF INTERVENTIONAL DEVICE ASSEMBLY, filed Nov. 30, 2023, which is incorporated herein in its entirety.

### Overview of Method

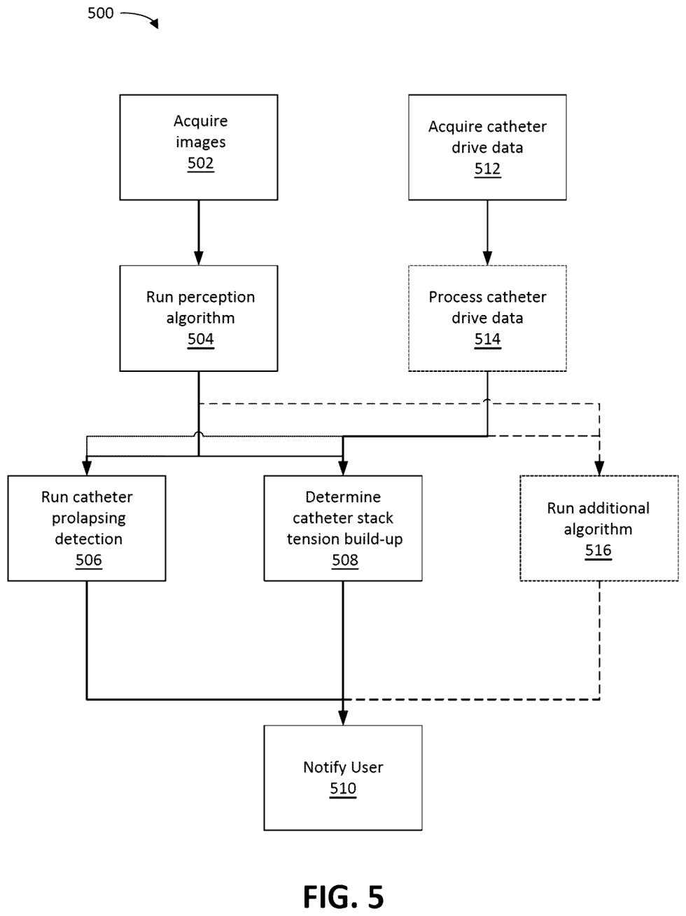

Methods in accordance with the present disclosure may be used while performing neurovascular procedures. FIG. 5 schematically diagrams an example method 500 of processing image data and catheter drive data to detect and/or predict catheter prolapse.

At step 502, acquire images. As discussed herein, the imaging system 116 can capture images of a patient. The images may be x-ray fluoroscopic images. In some examples, a live feed of the imaging system 116 can be streamed to the processor 202 such that the processor 202 acquires the images. In examples where the processor 202 is remote to the imaging system 116, the streaming of images may be of sufficiently low bandwidth that the processor 202 can receive the streamed images over an internet-based connection in real time. In some embodiments, the image quality may be variable. In some embodiments, the image quality may depend on the bandwidth available. Images may be acquired from the imaging system 116 at a downsampled rate. For instance, the imaging system 116 may create images at a first rate while the images are streamed to the processor 202 at a second, lower rate. As an illustrative example, the imaging system 116 may generate images at 30 Hz, and every other image is streamed to the processor 202, resulting in a downsampling of a factor of two to 15 Hz. It may be desirable to downsample for streaming to reduce bandwidth used when streaming images from the imaging system 116 to the processor 202.

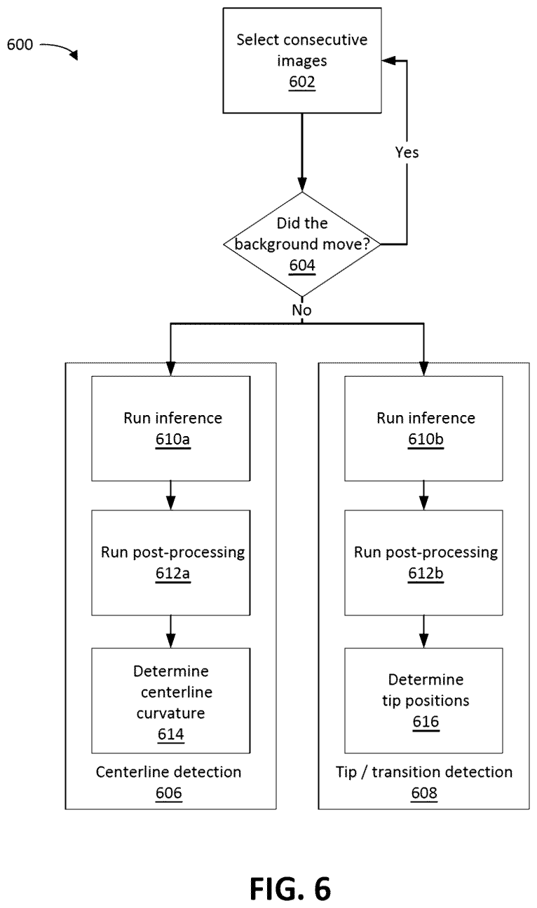

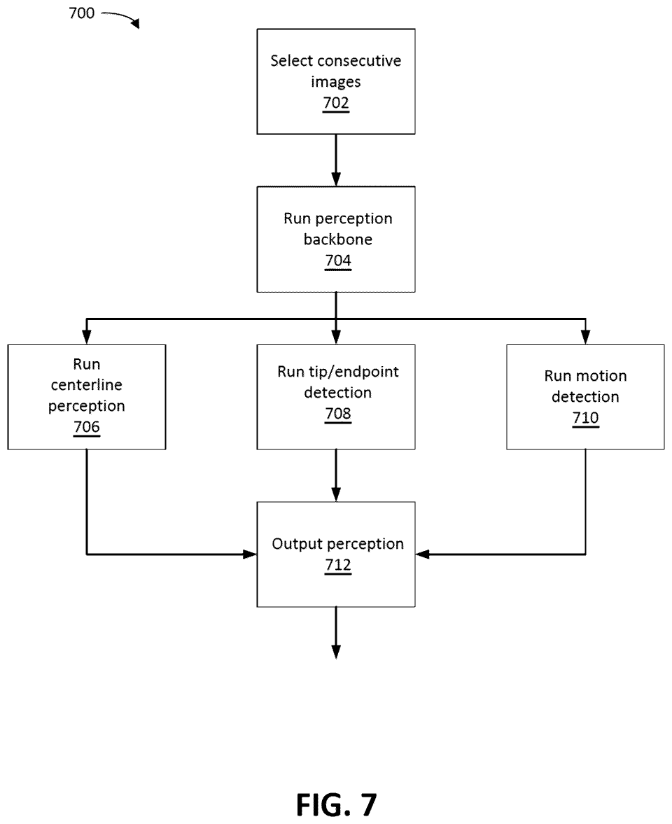

At step 504, run perception algorithm on the images. The processor 202 is capable of carrying out a perception algorithm in accordance with the present disclosure (for instance, as discussed with reference to FIGS. 6 and 7). The images received from the imaging system 116 may be input to such perception algorithms. The perception algorithm may be capable of estimating a catheter shaft position and/or a catheter tip position. In some examples, the processor 202 may down-sample the received images. As an illustrative example, the processor may receive images at 60 Hz and may process every other image, resulting in a downsampling of a factor of two to 30 Hz. Such downsampling may be desirable, for example, to match an image sampling frequency to a sampling frequency of catheter drive data. Additionally or alternatively, such downsampling may be desirable, for example, to reduce the processing power needed to process all the images.

In some examples, the perception algorithms can output a guidewire tip position, transition point position(s), and/or centerline position for each image. In some examples, the perception algorithms can output an estimated catheter shaft position and/or an estimated catheter tip position for each image subject to the perception algorithm.

At step 512, acquire catheter drive data. As discussed herein, the catheter drive system 118 can measure motion, change in position, force exerted on a catheter, or other parameters as provided for by the present disclosure. The catheter drive system 118 can transmit such data to the processor 202.

At step 514, optionally process catheter drive data. In some examples, the processor 202 is capable of processing the movement data received from the catheter drive system 118. Such processing may be desirable to, for example, convert and/or down sample the received catheter drive data to a frequency that is similar to that of the images. For instance, in examples where the images are captured at 30 Hz, it may be desirable to process the catheter drive data to convert it to a 30 Hz format. In some examples, it might be desirable to process the received catheter drive data such that the units of distance and/or force are the same as those with reference to the units used to indicate the catheter position as perceived by the algorithm(s) of step 504.

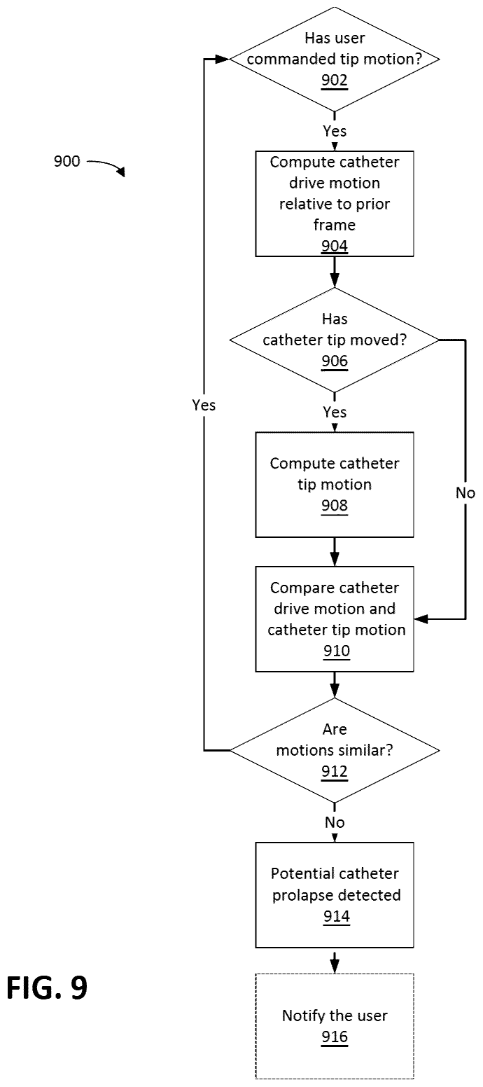

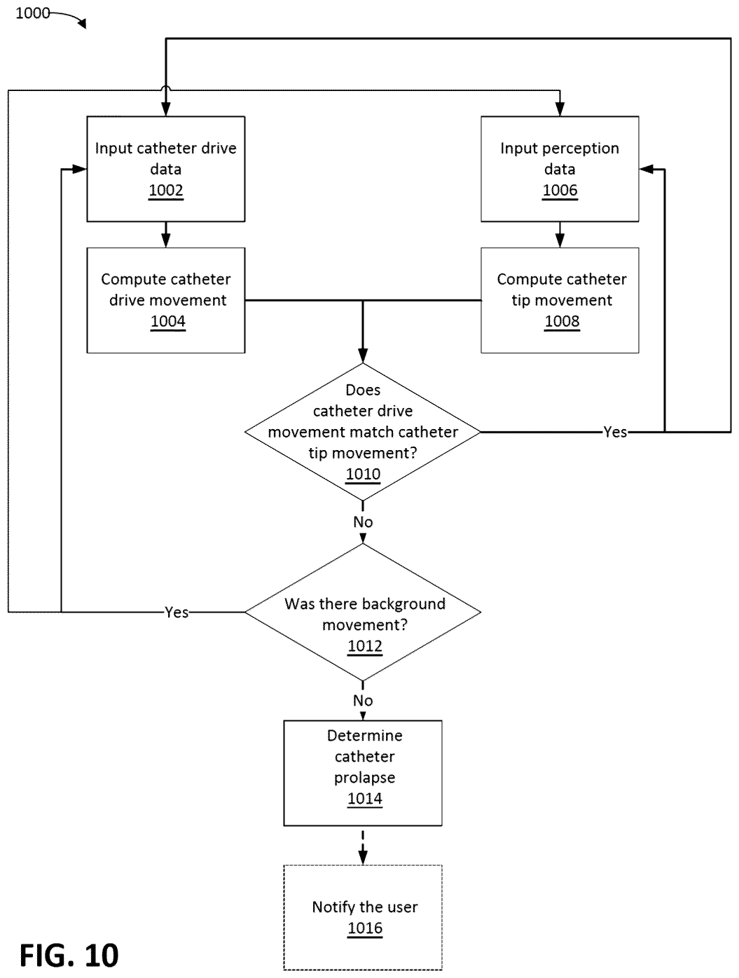

At step 506, run catheter prolapsing detection. Examples of catheter prolapsing detection methods are discussed with reference to FIGS. 9 and 10. The processor 202 can run a catheter prolapsing detection algorithm in accordance with the present disclosure. The inputs to such a catheter prolapsing detection algorithm may include the estimated catheter tip position and/or catheter centerline position and the catheter drive data (optionally, processed catheter drive data). Accordingly, the processor 202 can compare a change in the estimated catheter tip position and/or catheter centerline position to the catheter drive data. In situations where there is no catheter prolapse, movement of the catheter tip position and/or catheter centerline position may correspond closely to movement evidenced by the catheter drive data. In situations where there is catheter prolapse, movement of the catheter tip position and/or catheter centerline position may not closely correspond to movement evidenced by the catheter drive data. In some examples, if the difference in movement of the catheter tip position and/or catheter centerline position as compared to movement evidenced by the catheter drive data is above a threshold, the processor 202 can determine that catheter prolapse has occurred. Such a threshold may be an absolute threshold, a relative threshold, a threshold over time, a velocity threshold, or some combination thereof. In some embodiments, the processor 202 can determine the degree to which catheter prolapse has occurred. For instance, in a situation where there the catheter tip position has not been perceived to move based on the images, there may be a greater degree of catheter prolapse when the catheter drive data indicates a large expected motion as compared to a when the catheter drive data indicates only a small expected motion. A greater degree of catheter prolapse may be associated with a greater risk to the patient than that of a low degree of catheter prolapse.

At step 508, compute guidewire/catheter stack tension build-up. The processor 202 can determine a guidewire/catheter stack tension based on curvature of the catheter centerline as determined by the perception algorithm(s) discussed herein. Greater and/or sharper curvature may correspond to greater tension within the catheter. In some embodiments, the user may specify to the processor 202 the type of catheter(s) and/or guidewire being imaged so that such tension can be accurately determined. The processor 202 may additionally account for the location of transition points, as transition point location along the guidewire/catheter stack may affect the mechanics of the guidewire/catheter stack. The processor 202 can determine whether catheter prolapse is likely based at least in part on the tension of the catheter. In some examples, if the catheter tension is above a threshold, the processor 202 can determine that catheter prolapse is likely to occur. Such a threshold may be an absolute threshold, a relative threshold, a threshold over time, a velocity threshold, or some combination thereof.

At step 516, optionally run another algorithm that takes into account drive system data and catheter tip perception data. In some examples, the algorithm of step 516 may be an algorithm that accounts for a mapping of the patient's anatomy. In such examples, the mapping of the patient's anatomy may be compared to the catheter tip perception data and/or the drive system data to determine whether catheter prolapse has occurred and/or the extent to which prolapse has occurred. For example, if the catheter tip is perceived to be outside a mapped blood vessel of the patient, the algorithm may determine that prolapse has occurred. In some examples, the algorithm of step 516 may provide information pertaining to the surgical workflow or procedure. For example, the algorithm of step 516 may notify a user that the guidewire tip is approaching or has arrived at a particular anatomical feature of the patient. In other examples, the algorithm may be agnostic to patient anatomy and may take into account only perceived guidewire/catheter position and drive system data.

At step 510, notify the user. The processor 202 can cause the display 204 to indicate that prolapse has been detected, for example in response to catheter prolapse being detected in step 506. The processor 202 can cause the display 204 to indicate that a likelihood of catheter prolapse high (e.g., above a threshold), for example in response to a determination that prolapse is likely in step 508. In some embodiments, the processor 202 can cause the display 204 to indicate a degree to which catheter prolapse occurred. As an illustrative example, in situations where no or a minimal degree of catheter prolapse is detected, the processor 202 may cause the display 204 to display a green indicator. In situations where a moderate degree catheter prolapse is detected, the processor 202 may cause the display 204 to display a yellow indicator. In situations where a high degree of catheter prolapse is detected, the processor 202 may cause the display 204 to display a red indicator. Additionally or alternatively, the processor 202 can cause an alarm in response to prolapse being detected. Additionally or alternatively, the processor 202 can cause an alarm in response to a high likelihood (e.g., above a threshold) of catheter prolapse. In some embodiments, the processor 202 can cause the control system 206 to provide tactile feedback to user in response to detection of catheter prolapse. In some embodiments, the processor 202 can cause the control system 206 to provide tactile feedback to user in response to a determination of a high likelihood of catheter prolapse. In some embodiments, the processor 202 can influence the drive logic or drive mode of the catheter drive system 118. Such control of the drive logic or drive mode may advantageously prevent and/or reduce the chance of the guidewire tip springing forward as a result of tension buildup in the guidewire/catheter stack. In some embodiments, the processor 202 can prevent and/or inhibit further movement by the catheter drive system 118 in response to detection of catheter prolapse. In some embodiments, the processor 202 can stop and/or inhibit further movement by the catheter drive system 118 in response to a determination of a high likelihood of catheter prolapse. In some embodiments, the processor 202 can retract the guidewire in response to the detection of catheter prolapse. In some embodiments, the processor 202 can retract the guidewire in response to the determination of a high likelihood of catheter prolapse. In some embodiments, the processor 202 can slow the rate at which the catheter drive system 118 moves the guidewire in response to the detection of catheter prolapse. In some embodiments, the processor 202 can slow the rate at which the catheter drive system 118 guidewire in response to the determination of a high likelihood of catheter prolapse.

### Perception Algorithms

The present disclosure provides for methods and algorithms for perceiving a catheter (also referred to herein as “perception algorithms”) within images of the imaging system. The perception methods and algorithms discussed herein may be carried out by a processor, for example the processor 202. Such methods and algorithms may use machine vision. Such perception algorithms may be capable of detecting a catheter centerline, a catheter tip position, and/or transition point positions. Before being input to the perception algorithms, the images of the imaging system may be processed. In some embodiments, the processor 202 to the display 204 information generated by perception algorithms in accordance with the present disclosure. For example, the processor 202 can output catheter centerline position, guidewire tip position, transition point locations of the guidewire/catheter stack, and/or image background motion information. In some embodiments, the display 204 overlays information generated by the perception algorithms on images of the image system. The overlay may be on images that are native or processed. Additionally, other information can be provided to the perception algorithm. For instance, the perception algorithm can take into account x-ray image data, robotic drive data, and fluidics data. In some examples, the x-ray image data can include native and/or digital subtraction angiography (DSA) images. In some examples, the robotic drive data can include catheter motion data. In some examples, the fluidics data can include timing of a contrast agent injection, a saline injection, and/or application of vacuum.

FIG. 6 schematically diagrams an example method 600 of perceiving a guidewire/catheter stack. In some examples, a single model may be able to perform all of the steps of method 600. In some examples, more than one model can be used in combination to perform the step of method 600.

At step 602, select consecutive images. The selected images may be native images generated by an imaging system. In some examples, two or more images may be selected. In examples where images are downsampled, the consecutive images may be consecutive after downsampling.

At step 604, assess whether the background of the images moved. If the background did move, repeat step 602, selecting different consecutive images. If the background did not move, proceed to steps 606 and/or 608. In other embodiments, movement detected in the background may be used to correct and/or adjust movement detected in the catheter centerline, tip, and transition points. It is to be understood that, in some embodiments, any of steps 604, 606, and 608 can be performed simultaneously, sequentially, in parallel, etc. In particular examples, steps 606 and 608 may be performed in parallel.

At step 606, detect catheter and guidewire curvature by carrying out steps 610a, 612a, and/or 614.

At step 610a, run an inference step. The inference step 610a includes running the selected images through a first neural network that can perceive a catheter and guidewire centerline and/or a shape of the guidewire/catheter stack as a whole. The first neural network can determine an overall shape of the catheter. In some embodiments, the first neural network can accept an input of number of frames, height, and weight. In some embodiments, the first neural network can accept a plurality of consecutive image frames as input into a plurality of channels of input to the first neural network. In such embodiments, 2, 3, 4, 5, 6, 7, 8, 9, 10, 11, 12, 13, 14, 15, or 16 consecutive images may be input to the first neural network. In some embodiments, 8 consecutive images are input to the first neural network.

At step 612a, run post-processing on the catheter shape perceived by the first neural network in step 610a. In some examples, the post-processing can check whether the catheter shape perceived in step 610a is realistic. In some examples, the post-processing can verify whether the catheter shape perceived in step 610a has changed significantly with respect to the catheter shape of previous images. In such examples, extreme changes in perceived catheter shape from one frame to the next may indicate potential mis-perception of the catheter shape by the perception algorithm. In some embodiments, the output of step 612a is a catheter segmentation mask.

At step 614, determine the centerline curvature based on the post-processed catheter shape generated in step 612a.

At step 608, detect catheter and guidewire tip and/or transition point location by carrying out steps 610b, 612b, and/or 616. In some examples, step 606 and step 608 can be executed by two different models (e.g., in parallel or sequentially): a first model executing step 606 and a second model executing step 608. In such examples, the first model can be trained with a number of images where the centerline is manually annotated. Training centerlines output by the first model during training can be validated against such manual annotation. In other examples, the first model can output a training centerline which can be manually validated. Additionally, the second model can be trained with a number of images where a tip/transition point positions are manually annotated. Tip/transition point positions output by the model during training can be validated against such manual annotation. In other examples, the second model can output training tip/transition point positions which can be manually validated. In other examples, step 606 and step 608 can be executed by a single model (e.g., together or sequentially). In such examples, the single model can be trained with a number of images where the centerline and/or the tip/transition points are annotated. Training centerlines and training tip/transition points output by the single model can be validated against such manual annotation. In other examples, the single model can output training centerline and training tip/transition points which can be manually validated.

At step 610b, run an inference step. The inference step 610b includes running the selected images through a second neural network that can perceive a catheter and guidewire tips and/or transition points of the guidewire/catheter stack as a whole. The transition points may include locations along the guidewire/catheter centerline at which an inner component emerges. For example, the detectable transition points may include the transition from a first catheter to the guide wire, from a second catheter to the first catheter, from a third catheter to the second catheter, from a fourth catheter, and so forth. In some embodiments, the second neural network that can detect the guidewire tips and/or transition points of the guidewire/catheter stack of step 610b may use a determined centerline location, for example the centerline location as determined by step 614. In some embodiments, the algorithm may be able to identify one or more lengths along the centerline which correspond to one of the guidewire, the first catheter, the second catheter, the third catheter, the fourth catheter, etc.

At step 612b, run post-processing on the tip and/or transition points perceived by the neural network in step 610b. In some embodiments, post-processing may determine if tip and/or transition points perceived by the neural network in step 610b are realistic. For instance, a hardware processor executing post-processing may compare positions of the guidewire tip and/or transition points in consecutive frames to assess whether the velocity of the guidewire tip and/or transition points are above a realistic threshold and are therefore potentially mis-perceived by the perception algorithm. In some examples, the hardware processor executing post-processing can check whether a radio-opaque portion of the guide wire or catheter motion matches the perceived overall motion of the guidewire and/or catheter. In some examples, the hardware processor executing post-processing can check whether a radio-opaque portion of the guide wire or catheter motion matches the drive system data. In some examples, the hardware processor executing post-processing may determine if tip and/or transition points perceived by the by the neural network in step 610b are realistic based on drive system data indicating no input movement. In other words, if the drive system has not caused any movement, it would be expected that the tip and/or transition points would not move between selected images.

At step 616, determine the guidewire tip and/or transition point locations based on the post-processed tip and/or transition point locations generated in step 612b.

In some examples, the first and second neural networks may be a single neural network, where the single neural network is capable of perceiving both a centerline position and tip and/or transition point locations.

FIG. 7 schematically diagrams an example method 700 of perceiving a guidewire/catheter stack.

At step 702, select consecutive images. The selected images may be native images generated by an imaging system. In some examples, two or more images may be selected. In examples where images are downsampled, the consecutive images may be consecutive after downsampling.

At step 704, run perception backbone. Step 704 may include running the selected consecutive images through a neural network encoder (also referred to herein as a “vision encoder”) capable of processing the input images to a set of context vectors suitable for steps 706, 708, and/or 710.

At step 706, run centerline perception. Step 706 may include running the context vectors generated in step 704 through a decoder to produce an output sequence indicative of the position of the centerline of the guidewire/catheter stack.

At step 708, run tip/endpoint detection. Step 708 may include running the context vectors generated in step 704 through a decoder to produce an output sequence indicative of the positions of catheter and guidewire tip and/or transition points of the guidewire/catheter stack as a whole. The transition points may include locations along the guidewire/catheter centerline at which an inner component emerges. For example, the detectable transition points may include the transition from a first aspiration catheter to the guide wire, from a second aspiration catheter to the first aspiration catheter, from a third aspiration catheter to the second aspiration catheter, from a fourth aspiration catheter, and so forth. In some embodiments, the decoder may be able to indicate one or more lengths along the centerline (e.g., the centerline as detected in step 706) which correspond to one of the guidewire, the first aspiration catheter, the second aspiration catheter, the third aspiration catheter, the fourth aspiration catheter, etc.

At step 710, run motion detection. Step 708 may include running the context vectors generated in step 704 through a decoder to produce an output sequence indicative of image background motion.

At step 712, output perception. The perception can include catheter tip position, catheter centerline position, transition point position(s), and/or background movement. In some embodiments, when the background of the has images has moved, certain images may be excluded from the perception output. In some embodiments, if the image background did not move or moved insubstantially, certain images may be included in the perception output. In other embodiments, movement detected in the background may be used to correct and/or adjust movement perceived in the catheter centerline as part of step 706 or the tip and transition points of the guidewire/catheter stack as part of step 708.

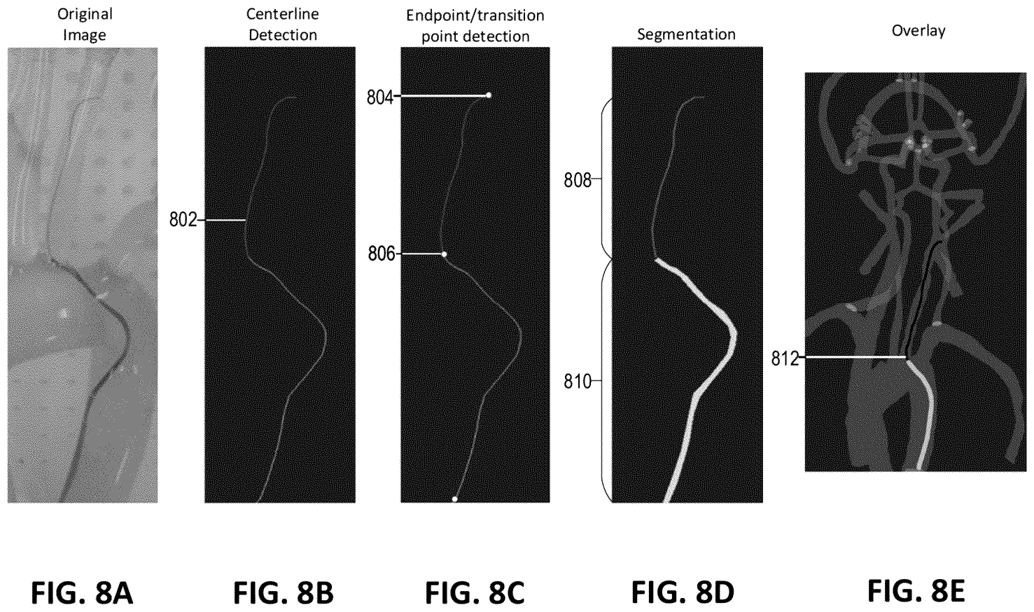

FIGS. 8A-8E show input and output of an example perception algorithm. FIG. 8A is an example image of a guidewire/catheter stack in a model blood vessel. FIG. 8B shows output of centerline detection, for example in accordance with steps 606 or 706 discussed herein, including the centerline 802 on a background. FIG. 8C shows output of endpoint/transition point detection, for example in accordance with steps 608 or 708 discussed herein. A point 804 indicates the guidewire tip. A point 806 indicates a transition between the catheter to the guidewire. FIG. 8D shows segmentation of the centerline based on the endpoint/transition point detection. A segment 808 represents the guidewire portion of the guidewire/catheter stack. A segment 810 represents the catheter portion of the guidewire/catheter stack. FIG. 8E shows an overlay of the perceived guidewire/catheter stack 812 on the mapped vasculature. In some examples, such mapping of a patient's vasculature may be generated from angiograms.

### Machine Learning and Artificial Intelligence

Methods discussed herein include use of neural networks, but it is to be understood that any suitable machine learning/artificial intelligence (AI) model may be suitably implemented. Different types of AI models may be utilized in an AI engine. In a first example, an AI model can be a transformer. A transformer is a deep learning architecture that relies on parallel multi-head attention mechanisms. An advantage of the transformer is that it generally requires less training time than other types of neural architectures. A Vision Transformer (ViT) adapts the Transformer structure initially for natural language processing to image and time series data like those acquired by the imaging system.

Inputs to the AI model can be configured, such as during a pre-processing step, a segmentation step, and/or a normalization step, to generate a processed input to the AI model. The processed input can be tailored for various objectives. Tailoring can include, but is not limited to segmenting or windowing image channels uniformly or differently based on an amount of time, based on signal features, based on time elapsed from a surgical event, based on another factor, the like or a combination thereof. The different processing of input can facilitate different amounts and/or types of attention to different aspects or features of the input signal and/or channels, resulting in different outputs, such as different risk assessments or distinctions based on different features and/or perspectives. One or more objectives can include, but are not limited to generating outputs based on different perspectives,

A neural network in accordance with the present disclosure can have any suitable architecture. In some examples, a neural network can be a deep neural network. In some examples, the deep neural network can include a triplet network architecture. The triplet network architecture may include three identical embedding networks, for example an anchor embedding network, a positive embedding network, and a negative embedding network. Non-limiting examples of the architecture of a deep neural network include a deep belief network architecture, a Boltzmann machine architecture, a restricted Boltzmann machine architecture, a deep Boltzmann machine architecture, or a deep auto-encoder architecture.

A deep neural network layer can apply linear or non-linear transformations to its input to generate its output. A deep neural network layer can be a normalization layer, a convolutional layer, a softsign layer, a rectified linear layer, a concatenation layer, a pooling layer, a recurrent layer, an inception-like layer, or any combination thereof. The normalization layer can normalize the brightness of its input to generate its output with, for example, L2 normalization. The normalization layer can, for example, normalize the brightness of a plurality of images with respect to one another at once to generate a plurality of normalized images as its output. Non-limiting examples of methods for normalizing brightness include local contrast normalization (LCN) or local response normalization (LRN). Local contrast normalization can normalize the contrast of an image non-linearly by normalizing local regions of the image on a per pixel basis to have mean of zero and variance of one (or other values of mean and variance). Local response normalization can normalize an image over local input regions to have mean of zero and variance of one (or other values of mean and variance). The normalization layer may speed up the computation of the embedding.

The number of the deep neural network layers in the deep neural network can be different in different implementations. For example, the number of the deep neural network can include 100 layers. The input type of a deep neural network layer can be different in different implementations. For example, a deep neural network layer can receive a training set T1 of (Img; Label) pairs as its input. As another example, a deep neural network layer can receive a triplet training set T2 of (ImgA; ImgP; ImgN). As yet another example, a deep neural network layer can receive the outputs of a number of deep neural network layers as its input. Image noise and other factors typical to real images can be added to the training set images before training the deep neural network.

The input of a deep neural network layer can be different in different implementations. For example, the input of a deep neural network layer can include the outputs of five deep neural network layers. As another example, the input of a deep neural network layer can include 1% of the deep neural network layers of the deep neural network. The output of a deep neural network layer can be the inputs of a number of deep neural layers. For example, the output of a deep neural network layer can be used as the inputs of five deep neural network layers. As another example, the output of a deep neural network layer can be used as the inputs of 1% of the deep neural network layers of the deep neural network layer.

The input size or the output size of a deep neural network layer can be large. The input size or the output size of a deep neural network layer can be n×m, where n denotes the width and m denotes the height of the input or the output. The channel sizes of the input or the output of a deep neural network layer can be different in different implementations. For example, the channel size of the input or the output of a deep neural network layer can be 32. The kernel size of a deep neural network layer can be different in different implementations. For example, the kernel size can be n×m, where n denotes the width and m denotes the height of the kernel. For example, n or m can be 5. The stride size of a deep neural network layer can be different in different implementations. For example, the stride size of a deep neural network layer can be 3.

### Catheter Prolapse Detection

The present disclosure provides for methods and algorithms for detecting catheter prolapse based on the guidewire/catheter stack as perceived by the algorithms and methods discussed herein and the drive system sensor data. The catheter prolapse detection algorithms herein may be carried out by a processor, for example the processor 202. The processor 202 may be capable of alerting a user of catheter prolapse. In some examples, the processor 202 can cause the display 204 to show a notification of catheter prolapse. In some examples, the processor 202 can cause an alarm. With reference to FIG. 5, such methods may be used in step 506.

FIG. 9 schematically diagrams an example method 900 for catheter prolapse detection.

At step 902, assess whether the user has commanded tip motion. In some examples, the processor 202 can receive data from the catheter drive system 118 regarding whether movement was inputted. In some examples where the processor 202 is in communication with a controller, the processor 202 may transmit movement commands to the catheter drive system 118 and thus the processor 202 can recognize when a movement command has been issued to the catheter drive system 118. If motion has been commanded, proceed. If motion has not been commanded, the assessment of step 902 can be repeated.

At step 904, compute catheter drive motion relative to the prior frame position. As discussed, data provided by the catheter drive system may include, among other things, motion, change in position, and/or force exerted on a catheter as provided for by the present disclosure. In step 904, the catheter drive motion can be computed based on one or more of these parameters. In some instances, the drive motion may be minimal such that no or only a minimal change in catheter tip is expected. Step 904 may include converting drive system sensor data to conform with images provided by the imaging system 116. In some examples, the drive system sensor data may be smoothed and/or outliers may be excluded. In some examples, the drive system sensor data may be measured at a frequency different than that of image acquisition by the imaging system. Such drive system data may additionally need to be summed, aggregated, converted, or otherwise processed such that the drive system data is more directly comparable to the images received from the imaging system.

At step 906, assess whether the catheter tip moved. The assessment of step 906 is based on analyzed images from the imaging system using the perception methods and algorithms provided herein. For example, with reference FIG. 6 or 7, the methods 600 or 700 may be used to perceive the catheter tip position. If the catheter tip has moved relative to a prior catheter tip position, proceed to step 908. If the catheter tip has not moved with respect to a prior catheter tip position, the method can proceed to step 910.

At step 908, compute the catheter tip motion. With reference to step 908, catheter tip motion is determined based on imaging. The perceived position of the catheter tip in step 906 can be compared to that of a previous frame to compute the catheter tip motion. The output of step 908 may be referred to as image-perceived motion.

At step 910, compare catheter drive motion to image-perceived motion. With reference to step 910, the comparison of catheter drive motion to motion perceived from the images essentially compares output of step 908 to output of step 904.

At step 912, assess whether the catheter drive motion is similar to the motion perceived from the images. With reference to step 912, the assessment may compare the catheter drive motion to the motion perceived from the images to generate a difference. If the difference is above a threshold, a determination can be made that the catheter drive motion is dissimilar to the motion perceived from the images. In some examples, the threshold may be an absolute threshold, a relative threshold, a threshold over time, a velocity threshold, or a combination thereof. In some examples, the thresholds may be in accordance with discussion step 1010 of FIG. 10 below. If the motions are similar, repeat step 902. If the motions are determined to be dissimilar, proceed to step 914.

At step 914, determine that catheter prolapse has been detected. Catheter prolapse may be detected where, based on output of step 912, the catheter drive motion and the motion perceived from the images are dissimilar.

At step 916, optionally notify the user of catheter prolapse. In some examples, a notification can be displayed to a user. In some examples, an alarm may be caused to indicate that catheter prolapse has been detected.

FIG. 10 schematically diagrams an example method 1000 for catheter prolapse detection.

At step 1002, input catheter drive data from the catheter drive system. Such data may be measured by various sensors of the catheter drive system discussed herein. As discussed, data provided by the catheter drive system may include, among other things, motion, change in position, and/or force exerted on a catheter as provided for by the present disclosure. As part of this step, the catheter drive data may be processed.

At step 1004, compute the expected catheter drive movement. With reference to step 1004, computation of catheter drive movement can include comparison of recent sensor drive data to previous catheter drive data to determine the change over the period of time between the acquisition of the recent sensor drive data and the previous catheter drive data. The expected catheter drive movement can correspond to the movement expected of the guidewire tip, guidewire/catheter stack centerline, transition point(s), and/or centerline shape assuming there is no prolapse. In some examples, the input catheter drive data can be converted to an expected catheter drive movement by a calibration conversion. That is to say, the drive system may be calibrated to allow data generated by the drive system to convertible to an actual distance moved by, for example, the guidewire tip.

At step 1006, input perception data. Such perception data may include, for example guidewire tip position, guidewire/catheter stack centerline position, transition point positions, and/or centerline shape. Such perception data may be generated using methods discussed herein, for example, with reference to FIGS. 6 and 7, methods 600 or 700. The perception data can be indicative of the perceived position of the guidewire tip and/or the catheter stack centerline, and transition point(s), and/or centerline shape as determined from the images.

At step 1008, compute catheter tip movement. With reference to step 1008, catheter tip movement is computed based on the perception data input in step 1006. That is to say, the catheter tip movement computed in step 1008 is responsive to actual movement of the guidewire/catheter stack within the patient's body, as step 1008 is based on imaging.

At step 1010, assess whether the catheter drive movement matches the catheter tip movement. With reference to step 1010, the assessment may compare the catheter drive movement computed in step 1004 (Δx_{drive}) to the catheter tip movement computed in step 1008 based on perception data (Δx_{image}) to generate a difference d (where d=|Δx_{drive}−Δx_{image}|). If the difference d is above a threshold t, a determination can be made that the catheter drive motion is dissimilar to the catheter tip movement. If, however, the motions match (i.e., are not dissimilar), repeat steps 1002 and 1006 with new and/or current catheter drive data and perception data. If the motions are determined to be dissimilar, proceed to step 1012. In some examples, the threshold t may be an absolute threshold t_{absolute}, a relative threshold tᵣₑₗₐₜᵢᵥₑ, a threshold over time, a velocity threshold t_{velocity}, or a combination thereof. In examples where the threshold is an absolute threshold, the threshold may be 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm, 6.0 mm, 6.1 mm, 6.2 mm, 6.3 mm, 6.4 mm, 6.5 mm, 6.6 mm, 6.7 mm, 6.8 mm, 6.9 mm, 7.0 mm, 7.1 mm, 7.2 mm, 7.3 mm, 7.4 mm, 7.5 mm, 7.6 mm, 7.7 mm, 7.8 mm, 7.9 mm, 8.0 mm, 8.1 mm, 8.2 mm, 8.3 mm, 8.4 mm, 8.5 mm, 8.6 mm, 8.7 mm, 8.8 mm, 8.9 mm, 9.0 mm, 9.1 mm, 9.2 mm, 9.3 mm, 9.4 mm, 9.5 mm, 9.6 mm, 9.7 mm, 9.8 mm, 9.9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm, or within a range defined by any two of the previous values, though other values may be suitably implemented. In examples where the threshold is a relative threshold tᵣₑₗₐₜᵢᵥₑ, an absolute difference in motion between expected motion based on drive system data Δx_{drive} and the observed motion from the image system data Δx_{image} may be divided by either of Δx_{drive} or Δx_{image} before being compared with the threshold to determine whether the expected and perceived motions match or are dissimilar. That is to say, the determination that Δx_{drive} and Δx_{image} are dissimilar can be made in situations where:

$$\frac{\vert Δ {x}_{d r i v e} - Δ {x}_{i m a g e} \vert}{Δ {x}_{i m a g e}} \geq {t}_{r elative} or \frac{\vert Δ {x}_{d r i v e} - Δ {x}_{i m a g e} \vert}{Δ {x}_{d r i v e}} \geq {t}_{relative}$$

In examples where the threshold is a relative threshold, the relative threshold tᵣₑₗₐₜᵢᵥₑ may be 0.5%, 0.75%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or within a range defined by any two of the previous values, though other values may be suitably implemented. In examples where the threshold is a velocity threshold, the velocity threshold t_{velocity} may be 0.05 mm/s, 0.1 mm/s, 0.2 mm/s, 0.3 mm/s, 0.4 mm/s, 0.5 mm/s, 0.6 mm/s, 0.7 mm/s, 0.8 mm/s, 0.9 mm/s, 1.0 mm/s, 1.1 mm/s, 1.2 mm/s, 1.3 mm/s, 1.4 mm/s, 1.5 mm/s, 1.6 mm/s, 1.7 mm/s, 1.8 mm/s, 1.9 mm/s, 2.0 mm/s, 2.1 mm/s, 2.2 mm/s, 2.3 mm/s, 2.4 mm/s, 2.5 mm/s, 2.6 mm/s, 2.7 mm/s, 2.8 mm/s, 2.9 mm/s, 3.0 mm/s, 3.1 mm/s, 3.2 mm/s, 3.3 mm/s, 3.4 mm/s, 3.5 mm/s, 3.6 mm/s, 3.7 mm/s, 3.8 mm/s, 3.9 mm/s, 4.0 mm/s, 4.1 mm/s, 4.2 mm/s, 4.3 mm/s, 4.4 mm/s, 4.5 mm/s, 4.6 mm/s, 4.7 mm/s, 4.8 mm/s, 4.9 mm/s, 5.0 mm/s, 5.1 mm/s, 5.2 mm/s, 5.3 mm/s, 5.4 mm/s, 5.5 mm/s, 5.6 mm/s, 5.7 mm/s, 5.8 mm/s, 5.9 mm/s, 6.0 mm/s, 6.1 mm/s, 6.2 mm/s, 6.3 mm/s, 6.4 mm/s, 6.5 mm/s, 6.6 mm/s, 6.7 mm/s, 6.8 mm/s, 6.9 mm/s, 7.0 mm/s, 7.1 mm/s, 7.2 mm/s, 7.3 mm/s, 7.4 mm/s, 7.5 mm/s, 7.6 mm/s, 7.7 mm/s, 7.8 mm/s, 7.9 mm/s, 8.0 mm/s, 8.1 mm/s, 8.2 mm/s, 8.3 mm/s, 8.4 mm/s, 8.5 mm/s, 8.6 mm/s, 8.7 mm/s, 8.8 mm/s, 8.9 mm/s, 9.0 mm/s, 9.1 mm/s, 9.2 mm/s, 9.3 mm/s, 9.4 mm/s, 9.5 mm/s, 9.6 mm/s, 9.7 mm/s, 9.8 mm/s, 9.9 mm/s, 10 mm/s, 11 mm/s, 12 mm/s, 13 mm/s, 14 mm/s, 15 mm/s, 16 mm/s, 17 mm/s, 18 mm/s, 19 mm/s, 20 mm/s, 21 mm/s, 22 mm/s, 23 mm/s, 24 mm/s, 25 mm/s, 26 mm/s, 27 mm/s, 28 mm/s, 29 mm/s, or 30 mm/s, or within a range defined by any two of the previous values, though other values may be suitably implemented.

At step 1012, assess whether there was background movement. Background movement includes at least movement of the patient's bed, movement of the imaging system, or movement of the patient. Background movement may confound or affect step 1010, making it more difficult to determine whether perceived movement in the guidewire/catheter stack is a result of the catheter drive system or due to movement in the bed, imaging system, or patient. In some examples, the assessment of background movement with reference to step 1012 may involve analyzing the background in images provided by the imaging system. In some embodiments, the assessment of background movement may involve receiving data from motion sensors of the bed and/or the imaging system. If there was no background movement, proceed to step 1014. If there was background movement, repeat steps 1002 and 1006 with new and/or current catheter drive data and perception data. In alternative examples, detected background movement can be used to calculate a corrected change in observed motion from the images that accounts for background movement. In some examples, a change in background Δx_{background} can be subtracted from an observed motion from the image system data Δx_{image} to create a corrected observed motion Δx_{image_corrected}.

At step 1014, determine catheter prolapse. Catheter prolapse may exist when, with reference to step 1010, catheter drive movement does not match observed guidewire and/or catheter tip movement and, with reference to step 1012. In some examples, catheter drive movement is deemed to be dissimilar to (i.e., not match) the catheter if a threshold is exceeded, with reference to step 1010. In some examples, this determination can only be made if there is no background movement. Alternatively, in examples where background movement is used to correct observed motion with reference to step 1012, catheter prolapse can be determined using a background motion-corrected change in observed motion from the images.

At step 1016, optionally notify the user of catheter prolapse. In some examples, a notification can be displayed to a user. In some such examples, the notification can require user acknowledgement before allowing the user to continue to use the drive system. In some examples, the user can be notified via haptic feedback in a controller. In some examples, an alarm may be caused to indicate catheter prolapse has been detected.

### Definitions

While the above detailed description has shown, described, and pointed out novel features as applied to various embodiments, it will be understood that various omissions, substitutions, and changes in the form and details of the systems, devices or methods illustrated can be made without departing from the spirit of the disclosure. As will be recognized, certain embodiments described herein can be embodied within a form that does not provide all of the features and benefits set forth herein, as some features can be used or practiced separately from others.

Many other variations than those described herein will be apparent from this disclosure. For example, depending on the embodiment, certain steps, acts, events, or functions of any of the algorithms described herein can be performed in a different sequence, can be added, merged, or left out altogether (e.g., not all described acts or events are necessary for the practice of the algorithms). Moreover, in certain embodiments, steps, acts, events, or functions can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

The system and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

As referred to herein, a “guidewire/catheter stack” refers to the structure formed by one or more catheters enveloping a guidewire.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

The term “and/or” herein has its broadest, least limiting meaning which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Although the foregoing disclosure has been described in terms of certain preferred embodiments, other embodiments will be apparent to those of ordinary skill in the art from the disclosure herein. Additionally, other combinations, omissions, substitutions and modifications will be apparent to the skilled artisan in view of the disclosure herein. Accordingly, the present invention is not intended to be limited by the description of the preferred embodiments, but is to be defined by reference to claims.

What is claimed is:

1. A system for detecting catheter or guidewire prolapse, the system comprising:
one or more processors configured to receive a plurality of images from an imaging system and drive system sensor data from a catheter drive system, the one or more processors further configured to:
determine a first catheter position based at least in part on a first of the plurality of images, the first catheter position comprising at least a first endpoint and a first centerline;
determine a second catheter position of based at least in part on a second of the plurality of images, the second catheter position comprising at least a second endpoint and a second centerline;
determine a visual position change based on a comparison of the first endpoint to the second endpoint;
determine an expected motion based at least in part of the drive system sensor data and a drive calibration, wherein the expected motion corresponds to drive system user input over a duration spanning the first of the plurality of images to the second of the plurality of images, the drive calibration comprising an expected movement distance corresponding to a calibration input;
determine a difference between the visual position change and the expected motion; and
cause an alert to a user of the system if the difference exceeds a prolapse threshold, the alert indicative of occurrence of catheter or guidewire prolapse.

2. The system of claim 1, wherein the prolapse threshold is a relative threshold of 5% difference between movement indicated from the images and from the drive system data.

3. The system of claim 1, wherein the alert comprises a user graphic.

4. The system of claim 3, wherein the user graphic comprises a prompt, wherein the prompt requires user input to allow further input to the catheter drive system.

5. The system of claim 1, wherein the alert comprises haptic feedback to a controller of the drive system.

6. The system of claim 1, further comprising the catheter drive system.

7. The system of claim 1, further comprising the imaging system.

8. The system of claim 1, wherein the first endpoint and second endpoint correspond to an endpoint of a guidewire in the first and second of the plurality of images.

9. The system of claim 1, wherein the first endpoint and second endpoint correspond to an endpoint of a catheter in the first and second of the plurality of images.

10. The system of claim 1, wherein the one or more processors is configured to execute programming to for computer vision analysis of at least some of the plurality of images.

11. The system of claim 1, wherein the one or more processors are further configured to:
compare the first centerline to the second centerline to determine a curvature change; and
cause a buckling alert to a user of the system if the curvature change exceeds a curvature threshold.

12. The system of claim 1, wherein at least some of the plurality of images comprise an image of a radio-opaque portion of the catheter or guidewire, and wherein the processor is configured to validate at least one of the determined first catheter position and second catheter position against a position of the radio-opaque portion of the catheter or guidewire.

13. A method of robotically controlling a catheter, the method comprising:
receiving a plurality of images from an imaging system and drive system sensor data from a catheter drive system;
determining a first catheter position based at least in part on a first of the plurality of images, the first catheter position comprising at least a first endpoint and a first centerline;
determining a second catheter position of based at least in part on a second of the plurality of images, the second catheter position comprising at least a second endpoint and a second centerline;
determining a visual position change based on a comparison of the first endpoint to the second endpoint;
determining an expected motion based at least in part of the drive system sensor data and a drive calibration, wherein the expected motion corresponds to drive system user input over a duration spanning the first of the plurality of images to the second of the plurality of images, the drive calibration comprising an expected movement distance corresponding to a calibration input;
determining a difference between the visual position change and the expected motion; and
causing an alert if the difference between the visual position change and the expected motion exceeds a prolapse threshold, the alert indicative of occurrence of catheter or guidewire prolapse.

14. The method of claim 13, wherein the prolapse threshold is 5%.

15. The method of claim 13, further comprising blocking user input to a catheter drive system if the difference between the visual position change and the expected motion exceeds the prolapse threshold.

16. The method of claim 15, wherein the alert comprises a prompt, and wherein the prompt requires user input to allow further input to the catheter drive system.

17. The method of claim 13, wherein the first endpoint and second endpoint correspond to an endpoint of a guidewire or a catheter in the first and second of the plurality of images.

18. The method of claim 13, comprising:
comparing the first centerline to the second centerline to determine a curvature change; and
causing a buckling alert if the curvature change exceeds a curvature threshold.

19. The method of claim 13, wherein the alert comprises haptic feedback to a controller of the drive system.

20. The method of claim 13, wherein at least some of the plurality of images comprise an image of a radio-opaque portion of the catheter or guidewire, and wherein the method further comprises validating at least one of the determined first catheter position and second catheter position against a position of the radio-opaque portion of the catheter or guidewire.
